(19) United States
(12) Patent Application Publication
(10) Pub. No.: US 2018/0021585 A1
(43) Pub. Date: Jan. 25, 2018

(54) LEADLESS MEDICAL SYSTEM FOR QUANTIFYING VENTRICLE TO VENTRICLE DYSSYNCHRONY

(71) Applicant: CARDIAC PACEMAKERS, INC., St. Paul, MN (US)

(72) Inventors: Qi An, Blaine, MN (US); Yinghong Yu, Shoreview, MN (US); Michael J. Kane, St. Paul, MN (US); Pramodsingh Hirasingh Thakur, Woodbury, MN (US); Jeffrey E. Stahmann, Ramsey, MN (US)

(73) Assignee: CARDIAC PACEMAKERS, INC., St. Paul, MN (US)

(21) Appl. No.: 15/654,342

(22) Filed: Jul. 19, 2017

Related U.S. Application Data
(60) Provisional application No. 62/364,642, filed on Jul. 20, 2016.

(51) Int. Cl. A61N 1/375; A61B 5/0215; A61N 1/365; A61N 1/37

(52) U.S. Cl. CPC A61N 1/3756; A61N 1/37; A61B 5/02158; A61N 1/36564

(57) ABSTRACT

A medical system includes two or more leadless implantable medical devices, each implanted in a different ventricle of the heart and each including a housing, a first electrode secured relative to the housing, a second electrode secured relative to the housing, and a pressure sensor secured relative to the housing. Each of the leadless implantable medical devices may further include circuitry in the housing operatively coupled to the corresponding first electrode, second electrode, and pressure sensor. The medical system may be configured to determine and store a plurality of pressure-pressure data pairs and/or impedance-impedance data pairs generated by the two or more leadless implantable medical devices, from which a representation of a pressure-pressure loop or volume-volume loop may be determined, to facilitate cardiac resynchronization therapy (CRT), patient health status monitoring, and/or the management of a non-CRT cardiac therapy.

### CROSS REFERENCE TO RELATED APPLICATIONS

This application claims the benefit of U.S. Provisional Patent Application Ser. No. 62/364,642 filed on Jul. 20, 2016, the disclosure of which is incorporated herein by reference.

### TECHNICAL FIELD

The present disclosure generally relates to implantable medical devices and more particularly to implantable medical devices for treating heart conditions.

### BACKGROUND

Medical devices are often used to treat patients suffering from various heart conditions. These heart conditions may lead to rapid, irregular, and/or inefficient heart contractions. To help alleviate some of these conditions, various devices (e.g., pacemakers, defibrillators, etc.) can be implanted in a patient's body. Such devices often monitor and/or provide therapy, such as electrical stimulation therapy, to the patient's heart to help the heart operate in a more normal, efficient and/or safe manner. In some cases, a patient may have multiple implanted devices that cooperate to monitor and/or provide therapy to the patient's heart.

### SUMMARY

The present disclosure generally relates to medical devices, and more particularly to implantable medical devices used for quantifying and/or treating ventricle to ventricle dyssynchrony.

One example medical system may include two or more leadless implantable medical devices, each implanted in a different ventricle of the heart and each including a housing, a first electrode secured relative to the housing, a second electrode secured relative to the housing, and a pressure sensor secured relative to the housing. Each of the leadless implantable medical devices may further include circuitry in the housing operatively coupled to the corresponding first electrode, second electrode, and pressure sensor. The medical system may be configured to determine and store a plurality of pressure-pressure data pairs and/or impedance-impedance data pairs generated by the two or more leadless implantable medical devices, from which a representation of a pressure-pressure loop or volume-volume loop may be determined, to facilitate cardiac resynchronization therapy (CRT), patient health status monitoring, and/or the management of a non-CRT cardiac therapy.

In another example medical system, a first leadless cardiac pacemaker (LCP) is used. The first LCP may comprise a housing, a first electrode secured relative to the housing and exposed to the environment outside of the housing, a second electrode secured relative to the housing and exposed to the environment outside of the housing, the second electrode is spaced from the first electrode, a pressure sensor secured relative to the housing and is coupled to the environment outside of the housing, and circuitry in the housing in communication with the first electrode, the second electrode, and the pressure sensor, the circuitry configured to determine at a first time during a cardiac cycle a first pressure via the pressure sensor. The system may further comprise a second LCP. The second LCP may comprise a housing, a first electrode secured relative to the housing and exposed to the environment outside of the housing, a second electrode secured relative to the housing and exposed to the environment outside of the housing, the second electrode is spaced from the first electrode, a pressure sensor secured relative to the housing and is coupled to the environment outside of the housing, and circuitry in the housing in communication with the first electrode, the second electrode, and the pressure sensor the circuitry configured to determine at the same first time as the circuitry in the first LCP a first pressure via the pressure sensor to generate a first pressure-pressure data pair.

Alternatively or additionally to any of the examples above, in another example, at least one of the circuitry of the first or second LCP may be configured to wirelessly transmit the first pressure-pressure data pair to a remote device.

Alternatively or additionally to any of the examples above, in another example, the circuitry of the first and second LCP may each be configured to determine, at a second time during the cardiac cycle, a second pressure via the corresponding pressure sensor, resulting in a second pressure-pressure data pair.

Alternatively or additionally to any of the examples above, in another example, the first time may correspond to an S1 heart sound and the second time corresponds to an S2 heart sound.

Alternatively or additionally to any of the examples above, in another example, the circuitry of the first and second LCP may each be further configured to determine, at a plurality of times between the first time and the second time, a plurality of corresponding pressures via the corresponding pressure sensor, resulting in a plurality of additional pressure-pressure data pairs.

Alternatively or additionally to any of the examples above, in another example, at least one of the circuitry of the first or second LCP may be further configured to wirelessly transmit the first pressure-pressure data pair, the second pressure-pressure data pair, and the plurality of additional pressure-pressure data pairs to a remote device.

Alternatively or additionally to any of the examples above, in another example, the remote device may be configured to generate and display a pressure-pressure loop that is based at least in part on the first pressure-pressure data pair, the second pressure-pressure data pair, and the plurality of additional pressure-pressure data pairs.

Alternatively or additionally to any of the examples above, in another example, the remote device may be configured to store and display a plurality of pressure-pressure loops generated over a period of time.

Alternatively or additionally to any of the examples above, in another example, at least one of the circuitry of the first or second LCP may be further configured to record contextual data indicating the patient's physiological state present during the cardiac cycle.

Alternatively or additionally to any of the examples above, in another example, the indication of the patient's physiological state may comprise one or more of a level of activity, sleep/wakefulness, a disease status, an effect of a therapy, a heart rate, a respiratory rate, a blood gas, a blood analyte, or a posture.

Alternatively or additionally to any of the examples above, in another example, the circuitry of the first and second LCP may each be further configured to average the first pressures over a plurality of cardiac cycles, resulting in an averaged first pressure-pressure data pair for the first time during an averaged cardiac cycle.

Alternatively or additionally to any of the examples above, in another example, the circuitry of the first LCP may be configured to determine an impedance between the first and second electrodes of the first LCP at each of a plurality of times during the cardiac cycle, the circuitry of the second LCP may be configured to determine an impedance between the first and second electrodes of the second LCP at each of the plurality of times during the cardiac cycle to generate a plurality of impedance-impedance data pairs and at least one of the circuitry of the first or second LCP may be configured to wirelessly transmit the plurality of impedance-impedance data pairs to a remote device.

In another example, a leadless medical device may comprise a housing, a first electrode secured relative to the housing and exposed to the environment outside of the housing, a second electrode secured relative to the housing and exposed to the environment outside of the housing, the second electrode may be spaced from the first electrode, a pressure sensor secured relative to the housing and is coupled to the environment outside of the housing, and circuitry in the housing in operatively coupled to the first electrode, the second electrode, and the pressure sensor, the circuitry may be configured to determine a pressure via the pressure sensor at a plurality of times during a cardiac cycle of a patient to generate a plurality of time/pressure pairs that correspond to the cardiac cycle, the circuitry further configured to wirelessly communicate the plurality of time/pressure pairs to a remote device via the first electrode and the second electrode.

Alternatively or additionally to any of the examples above, in another example, the circuitry may receive a synchronization marker from the remote device, and the plurality of times are expressed relative to the synchronization maker.

Alternatively or additionally to any of the examples above, in another example, the circuitry may further wirelessly communicate a synchronization marker to the remote device, and the plurality of times are expressed relative to the synchronization maker.

In another example, a method may comprise receiving a measure of right ventricle pressure from a first leadless medical device implanted in the right ventricle (RV) of a patient's heart and a measure of left ventricle pressure from a second leadless medical device implanted in the left ventricle (LV) of the patient's heart for each of a plurality of times during a cardiac cycle, and generating a pressure-pressure loop that is based at least in part on the measure of right ventricle pressure and the measure of left ventricle pressure for each of the plurality of times during the cardiac cycle, and displaying the pressure-pressure loop on a display and/or altering an operation of the first leadless medical device and/or the second leadless medical device based at least in part on the pressure-pressure loop.

Alternatively or additionally to any of the examples above, in another example, the first leadless medical device and the second leadless medical device may be leadless cardiac pacemakers, and the method may further comprise changing one or more pacing parameters of the first leadless medical device and/or one or more pacing parameters of the second leadless medical device to improve synchronization between the RV and the LV of the patient's heart.

Alternatively or additionally to any of the examples above, in another example, the first leadless medical device and the second leadless medical device may be leadless cardiac pacemakers, and the method may further comprise changing an in implant site of the first leadless medical device and/or the implant site of the second leadless medical device to improve synchronization between the RV and the LV of the patient's heart.

Alternatively or additionally to any of the examples above, in another example, the cardiac cycle may be an averaged cardiac cycle, and the measure of right ventricle pressure and the measure of left ventricle pressure are averaged pressures for each of the plurality of times during the averaged cardiac cycle.

Alternatively or additionally to any of the examples above, in another example, the method may further comprise receiving contextual data indicating a patient's physiological state present during the cardiac cycle, and wherein the patient's physiological state comprises one or more of a level of activity, sleep/wakefulness, a disease status, an effect of a therapy, a heart rate, a respiratory rate, a blood gas, a blood analyte, or a posture. The above summary is not intended to describe each embodiment or every implementation of the present disclosure. Advantages and attainments, together with a more complete understanding of the disclosure, will become apparent and appreciated by referring to the following description and claims taken in conjunction with the accompanying drawings.

### BRIEF DESCRIPTION OF THE DRAWINGS

The disclosure may be more completely understood in consideration of the following description of various illustrative embodiments in connection with the accompanying drawings, in which:

FIG. 1 is a schematic block diagram of an illustrative leadless cardiac pacemaker (LCP) according to one example of the present disclosure;

FIG. 2 is a schematic block diagram of another medical device (MD), which may be used in conjunction with an LCP 100 (FIG. 1) in order to detect and/or treat cardiac arrhythmias and other heart conditions;

FIG. 3 is a schematic diagram of an exemplary medical system that includes multiple LCPs and/or other devices in communication with one another;

FIG. 4 is a schematic diagram of an exemplary medical system that includes an LCP and another medical device, in accordance with yet another example of the present disclosure;

FIG. 5 is a schematic diagram of an exemplary medical system that includes an LCP and another medical device, in accordance with yet another example of the present disclosure;

FIG. 6 is a side view of an illustrative implantable leadless cardiac pacing device;

FIG. 7A is a plan view of an exemplary medical system including multiple leadless cardiac pacing devices implanted within a heart during ventricular filling;

FIG. 7B is a plan view of an exemplary medical system including multiple leadless cardiac pacing devices implanted within a heart during ventricular contraction;

FIG. 8A is a graph showing example pressures and volumes within the heart over time;

FIG. 8B is a graph showing an example electrocardiogram and heart sounds over time;

FIG. 9A is an illustrative pressure-pressure loop for the ventricles of a human heart;

FIG. 9B is an illustrative volume-volume loop for the ventricles of a human heart;

FIG. 10 is a flow diagram of an illustrative method for generating a pressure-pressure loop for the ventricles of a human heart;

FIG. 11 is a flow diagram of an illustrative method for generating a volume-volume loop for the ventricles of a human heart; and

FIG. 12 is a diagram illustrating the various uses of a pressure-pressure loop and/or volume-volume loop for the ventricles.

While the disclosure is amenable to various modifications and alternative forms, specifics thereof have been shown by way of example in the drawings and will be described in detail. It should be understood, however, that the intention is not to limit aspects of the disclosure to the particular illustrative embodiments described. On the contrary, the intention is to cover all modifications, equivalents, and alternatives falling within the spirit and scope of the disclosure.

### DESCRIPTION

The following description should be read with reference to the drawings in which similar elements in different drawings are numbered the same. The description and the drawings, which are not necessarily to scale, depict illustrative embodiments and are not intended to limit the scope of the disclosure.

A normal, healthy heart induces contraction by conducting intrinsically generated electrical signals throughout the heart. These intrinsic signals cause the muscle cells or tissue of the heart to contract. This contraction forces blood out of and into the heart, providing circulation of the blood throughout the rest of the body. However, many patients suffer from cardiac conditions that affect this contractility of their hearts. For example, some hearts may develop diseased tissues that no longer generate or conduct intrinsic electrical signals. In some examples, diseased cardiac tissues conduct electrical signals at differing rates, thereby causing an unsynchronized and inefficient contraction of the heart. In other examples, a heart may initiate intrinsic signals at such a low rate that the heart rate becomes dangerously low. In still other examples, a heart may generate electrical signals at an unusually high rate. In some cases such an abnormality can develop into a fibrillation state, where the contraction of the patient's heart chambers are almost completely de-synchronized and the heart pumps very little to no blood. Implantable medical devices, which may be configured to determine occurrences of such cardiac abnormalities or arrhythmias and deliver one or more types of electrical stimulation therapy to patient's hearts, may help to terminate or alleviate these and other cardiac conditions.

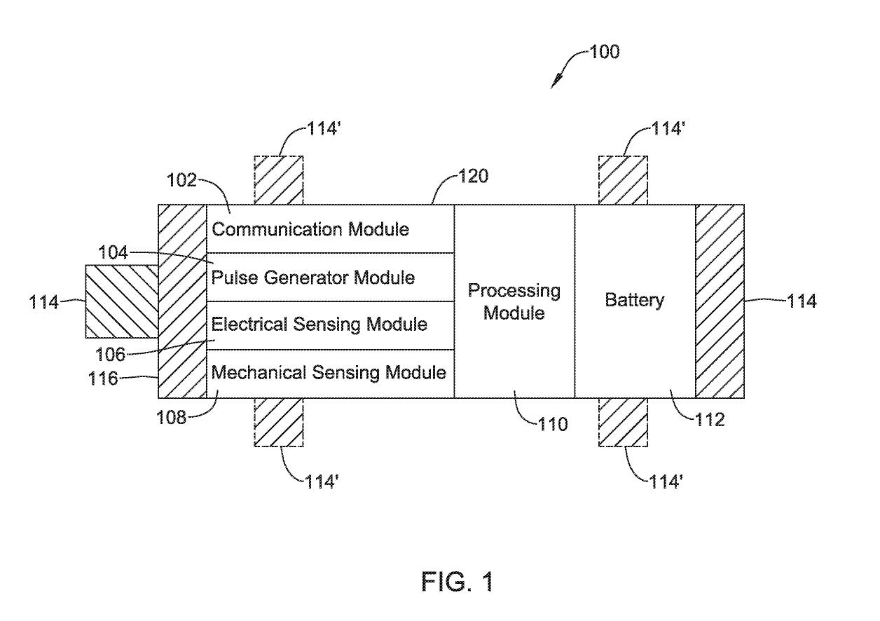

FIG. 1 depicts an illustrative leadless cardiac pacemaker (LCP) that may be implanted into a patient and may operate to prevent, control, or terminate cardiac arrhythmias in patients by, for example, appropriately employing one or more therapies (e.g. anti-tachycardia pacing (ATP) therapy, cardiac resynchronization therapy (CRT), bradycardia therapy, defibrillation pulses, and/or the like). As can be seen in FIG. 1, the LCP 100 may be a compact device with all components housed within the LCP 100 or directly on the housing 120. In the example shown in FIG. 1, the LCP 100 may include a communication module 102, a pulse generator module 104, an electrical sensing module 106, a mechanical sensing module 108, a processing module 110, a battery 112, and electrodes 114. The LCP 100 may include more or less modules, depending on the application.

The communication module 102 may be configured to communicate with devices such as sensors, other medical devices, and/or the like, that are located externally to the LCP 100. Such devices may be located either external or internal to the patient's body. Irrespective of the location, remote devices (i.e. external to the LCP 100 but not necessarily external to the patient's body) can communicate with the LCP 100 via the communication module 102 to accomplish one or more desired functions. For example, the LCP 100 may communicate information, such as sensed electrical signals, data, instructions, messages, etc., to an external medical device through the communication module 102. The external medical device may use the communicated signals, data, instructions and/or messages to perform various functions, such as determining occurrences of arrhythmias, delivering electrical stimulation therapy, storing received data, analyzing received data, and/or performing any other suitable function. The LCP 100 may additionally receive information such as signals, data, instructions and/or messages from the external medical device through the communication module 102, and the LCP 100 may use the received signals, data, instructions and/or messages to perform various functions, such as determining occurrences of arrhythmias, delivering electrical stimulation therapy, storing received data, analyzing received data, and/or performing any other suitable function. The communication module 102 may be configured to use one or more methods for communicating with remote devices. For example, the communication module 102 may communicate via radiofrequency (RF) signals, inductive coupling, optical signals, acoustic signals, conducted communication signals, and/or any other signals suitable for communication.

In the example shown in FIG. 1, the pulse generator module 104 may be electrically connected to the electrodes 114. In some examples, the LCP 100 may include one or more additional electrodes 114′. In such examples, the pulse generator 104 may also be electrically connected to the additional electrodes 114′. The pulse generator module 104 may be configured to generate electrical stimulation signals. For example, the pulse generator module 104 may generate electrical stimulation signals by using energy stored in a battery 112 within the LCP 100 and deliver the generated electrical stimulation signals via the electrodes 114 and/or 114′. Alternatively, or additionally, the pulse generator 104 may include one or more capacitors, and the pulse generator 104 may charge the one or more capacitors by drawing energy from the battery 112. The pulse generator 104 may then use the energy of the one or more capacitors to deliver the generated electrical stimulation signals via the electrodes 114 and/or 114′. In at least some examples, the pulse generator 104 of the LCP 100 may include switching circuitry to selectively connect one or more of the electrodes 114 and/or 114′ to the pulse generator 104 in order to select which of the electrodes 114/114′ (and/or other electrodes) the pulse generator 104 delivers the electrical stimulation therapy. The pulse generator module 104 may generate electrical stimulation signals with particular features or in particular sequences in order to provide one or multiple of a number of different stimulation therapies. For example, the pulse generator module 104 may be configured to generate electrical stimulation signals to provide electrical stimulation therapy to combat bradycardia, tachycardia, cardiac synchronization, bradycardia arrhythmias, tachycardia arrhythmias, fibrillation arrhythmias, cardiac synchronization arrhythmias and/or to produce any other suitable electrical stimulation therapy. Some more common electrical stimulation therapies include anti-tachycardia pacing (ATP) therapy, cardiac resynchronization therapy (CRT), and cardioversion/defibrillation therapy.

In some examples, the LCP 100 may not include a pulse generator 104 or may turn off the pulse generator 104. When so provided, the LCP 100 may be a diagnostic only device. In such examples, the LCP 100 may not deliver electrical stimulation therapy to a patient. Rather, the LCP 100 may collect data about cardiac electrical activity and/or physiological parameters of the patient and communicate such data and/or determinations to one or more other medical devices via the communication module 102.

In some examples, the LCP 100 may include an electrical sensing module 106, and in some cases, a mechanical sensing module 108. The electrical sensing module 106 may be configured to sense the cardiac electrical activity of the heart. For example, the electrical sensing module 106 may be connected to the electrodes 114/114′, and the electrical sensing module 106 may be configured to receive cardiac electrical signals conducted through the electrodes 114/114′. The cardiac electrical signals may represent local information from the chamber in which the LCP 100 is implanted. For instance, if the LCP 100 is implanted within a ventricle of the heart, cardiac electrical signals sensed by the LCP 100 through the electrodes 114/114′ may represent ventricular cardiac electrical signals. The mechanical sensing module 108 may include one or more sensors, such as an accelerometer, a blood pressure sensor, a heart sound sensor, a blood-oxygen sensor, a temperature sensor, a flow sensor and/or any other suitable sensors that are configured to measure one or more mechanical and/or chemical parameters of the patient. Both the electrical sensing module 106 and the mechanical sensing module 108 may be connected to a processing module 110, which may provide signals representative of the sensed mechanical parameters. Although described with respect to FIG. 1 as separate sensing modules, in some cases, the electrical sensing module 106 and the mechanical sensing module 108 may be combined into a single sensing module, as desired.

The electrodes 114/114′ can be secured relative to the housing 120 but exposed to the tissue and/or blood surrounding the LCP 100. In some cases, the electrodes 114 may be generally disposed on either end of the LCP 100 and may be in electrical communication with one or more of the modules 102, 104, 106, 108, and 110. The electrodes 114/114′ may be supported by the housing 120, although in some examples, the electrodes 114/114′ may be connected to the housing 120 through short connecting wires such that the electrodes 114/114′ are not directly secured relative to the housing 120. In examples where the LCP 100 includes one or more electrodes 114′, the electrodes 114′ may in some cases be disposed on the sides of the LCP 100, which may increase the number of electrodes by which the LCP 100 may sense cardiac electrical activity, deliver electrical stimulation and/or communicate with an external medical device. The electrodes 114/114′ can be made up of one or more biocompatible conductive materials such as various metals or alloys that are known to be safe for implantation within a human body. In some instances, the electrodes 114/114′ connected to LCP 100 may have an insulative portion that electrically isolates the electrodes 114/114′ from adjacent electrodes, the housing 120, and/or other parts of the LCP 100.

The processing module 110 can be configured to control the operation of the LCP 100. For example, the processing module 110 may be configured to receive electrical signals from the electrical sensing module 106 and/or the mechanical sensing module 108. Based on the received signals, the processing module 110 may determine, for example, occurrences and, in some cases, types of arrhythmias. Based on any determined arrhythmias, the processing module 110 may control the pulse generator module 104 to generate electrical stimulation in accordance with one or more therapies to treat the determined arrhythmia(s). The processing module 110 may further receive information from the communication module 102. In some examples, the processing module 110 may use such received information to help determine whether an arrhythmia is occurring, determine a type of arrhythmia, and/or to take particular action in response to the information. The processing module 110 may additionally control the communication module 102 to send/receive information to/from other devices.

In some examples, the processing module 110 may include a pre-programmed chip, such as a very-large-scale integration (VLSI) chip and/or an application specific integrated circuit (ASIC). In such embodiments, the chip may be pre-programmed with control logic in order to control the operation of the LCP 100. By using a pre-programmed chip, the processing module 110 may use less power than other programmable circuits (e.g. general purpose programmable microprocessors) while still being able to maintain basic functionality, thereby potentially increasing the battery life of the LCP 100. In other examples, the processing module 110 may include a programmable microprocessor. Such a programmable microprocessor may allow a user to modify the control logic of the LCP 100 even after implantation, thereby allowing for greater flexibility of the LCP 100 than when using a pre-programmed ASIC. In some examples, the processing module 110 may further include a memory, and the processing module 110 may store information on and read information from the memory. In other examples, the LCP 100 may include a separate memory (not shown) that is in communication with the processing module 110, such that the processing module 110 may read and write information to and from the separate memory.

The battery 112 may provide power to the LCP 100 for its operations. In some examples, the battery 112 may be a non-rechargeable lithium-based battery. In other examples, a non-rechargeable battery may be made from other suitable materials, as desired. Because the LCP 100 is an implantable device, access to the LCP 100 may be limited after implantation. Accordingly, it is desirable to have sufficient battery capacity to deliver therapy over a period of treatment such as days, weeks, months, years or even decades. In some instances, the battery 112 may a rechargeable battery, which may help increase the useable lifespan of the LCP 100. In still other examples, the battery 112 may be some other type of power source, as desired.

To implant the LCP 100 inside a patient's body, an operator (e.g., a physician, clinician, etc.), may fix the LCP 100 to the cardiac tissue of the patient's heart. To facilitate fixation, the LCP 100 may include one or more anchors 116. The anchor 116 may include any one of a number of fixation or anchoring mechanisms. For example, the anchor 116 may include one or more pins, staples, threads, screws, helix, tines, and/or the like. In some examples, although not shown, the anchor 116 may include threads on its external surface that may run along at least a partial length of the anchor 116. The threads may provide friction between the cardiac tissue and the anchor to help fix the anchor 116 within the cardiac tissue. In other examples, the anchor 116 may include other structures such as barbs, spikes, or the like to facilitate engagement with the surrounding cardiac tissue.

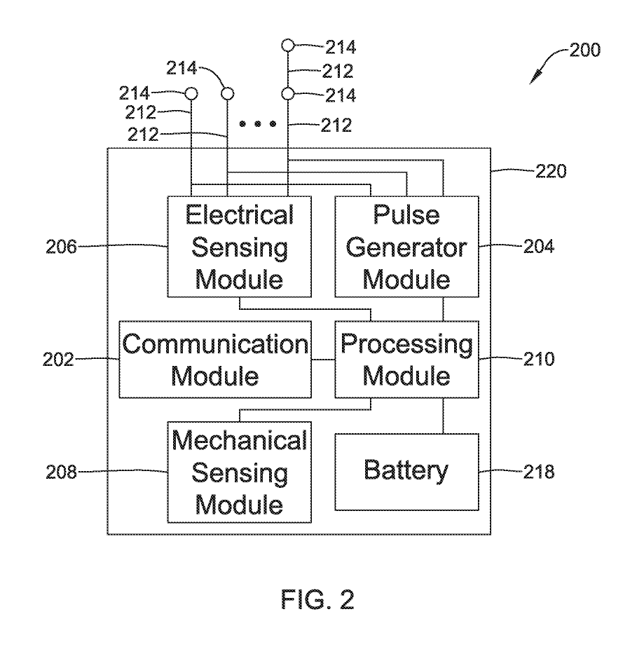

FIG. 2 depicts an example of another medical device (MD) 200, which in some cases may be used in conjunction with an LCP 100 (FIG. 1) in order to detect and/or treat cardiac arrhythmias and other heart conditions. In the example shown, the MD 200 may include a communication module 202, a pulse generator module 204, an electrical sensing module 206, a mechanical sensing module 208, a processing module 210, and a battery 218. Each of these modules may be similar to the modules 102, 104, 106, 108, and 110 of the LCP 100. Additionally, the battery 218 may be similar to the battery 112 of the LCP 100. In some examples, the MD 200 may have a larger volume within the housing 220 than LCP 100. In such examples, the MD 200 may include a larger battery and/or a larger processing module 210 capable of handling more complex operations than the processing module 110 of the LCP 100.

While it is contemplated that the MD 200 may be another leadless device such as shown in FIG. 1, in some instances the MD 200 may include leads such as leads 212. The leads 212 may include electrical wires that conduct electrical signals between the electrodes 214 and one or more modules located within the housing 220. In some cases, the leads 212 may be connected to and extend away from the housing 220 of the MD 200. In some examples, the leads 212 are implanted on, within, or adjacent to a heart of a patient. The leads 212 may contain one or more electrodes 214 positioned at various locations on the leads 212, and in some cases at various distances from the housing 220. Some of the leads 212 may only include a single electrode 214, while other leads 212 may include multiple electrodes 214. Generally, the electrodes 214 are positioned on the leads 212 such that when the leads 212 are implanted within the patient, one or more of the electrodes 214 are positioned to perform a desired function. In some cases, the one or more of the electrodes 214 may be in contact with the patient's cardiac tissue. In some cases, the one or more of the electrodes 214 may be positioned subcutaneously but adjacent the patient's heart. In some cases, the electrodes 214 may conduct intrinsically generated electrical signals to the leads 212, e.g. signals representative of intrinsic cardiac electrical activity. The leads 212 may, in turn, conduct the received electrical signals to one or more of the modules 202, 204, 206, and 208 of the MD 200. In some cases, the MD 200 may generate electrical stimulation signals, and the leads 212 may conduct the generated electrical stimulation signals to the electrodes 214. The electrodes 214 may then conduct the electrical signals and delivery the signals to the patient's heart (either directly or indirectly).

The mechanical sensing module 208, as with the mechanical sensing module 108, may contain or be electrically connected to one or more sensors, such as accelerometers, blood pressure sensors, heart sound sensors, blood-oxygen sensors, and/or other sensors which are configured to measure one or more mechanical/chemical parameters of the heart and/or patient. In some examples, one or more of the sensors may be located on the leads 212, but this is not required. In some examples, one or more of the sensors may be located in the housing 220.

While not required, in some examples, the MD 200 may be an implantable medical device. In such examples, the housing 220 of the MD 200 may be implanted in, for example, a transthoracic region of the patient. The housing 220 may generally include any of a number of known materials that are safe for implantation in a human body and may, when implanted, hermetically seal the various components of the MD 200 from fluids and tissues of the patient's body.

In some cases, the MD 200 may be an implantable cardiac pacemaker (ICP). In this example, the MD 200 may have one or more leads, for example leads 212, which are implanted on or within the patient's heart. The one or more leads 212 may include one or more electrodes 214 that are in contact with cardiac tissue and/or blood of the patient's heart. The MD 200 may be configured to sense intrinsically generated cardiac electrical signals and determine, for example, one or more cardiac arrhythmias based on analysis of the sensed signals. The MD 200 may be configured to deliver CRT, ATP therapy, bradycardia therapy, and/or other therapy types via the leads 212 implanted within the heart. In some examples, the MD 200 may additionally be configured provide defibrillation therapy.

In some instances, the MD 200 may be an implantable cardioverter-defibrillator (ICD). In such examples, the MD 200 may include one or more leads implanted within a patient's heart. The MD 200 may also be configured to sense cardiac electrical signals, determine occurrences of tachyarrhythmias based on the sensed signals, and may be configured to deliver defibrillation therapy in response to determining an occurrence of a tachyarrhythmia. In some instances, the MD 200 may be a subcutaneous implantable cardioverter-defibrillator (S-ICD). In examples where the MD 200 is an S-ICD, one of the leads 212 may be a subcutaneously implanted lead. In at least some examples where the MD 200 is an S-ICD, the MD 200 may include only a single lead which is implanted subcutaneously, but this is not required. In some cases, the S-ICD lead may extend subcutaneously from the S-ICD can, around the sternum and may terminate adjacent the interior surface of the sternum.

In some examples, the MD 200 may not be an implantable medical device. Rather, the MD 200 may be a device external to the patient's body, and may include skin-electrodes that are placed on a patient's body. In such examples, the MD 200 may be able to sense surface electrical signals (e.g. cardiac electrical signals that are generated by the heart or electrical signals generated by a device implanted within a patient's body and conducted through the body to the skin). In such examples, the MD 200 may be configured to deliver various types of electrical stimulation therapy, including, for example, defibrillation therapy.

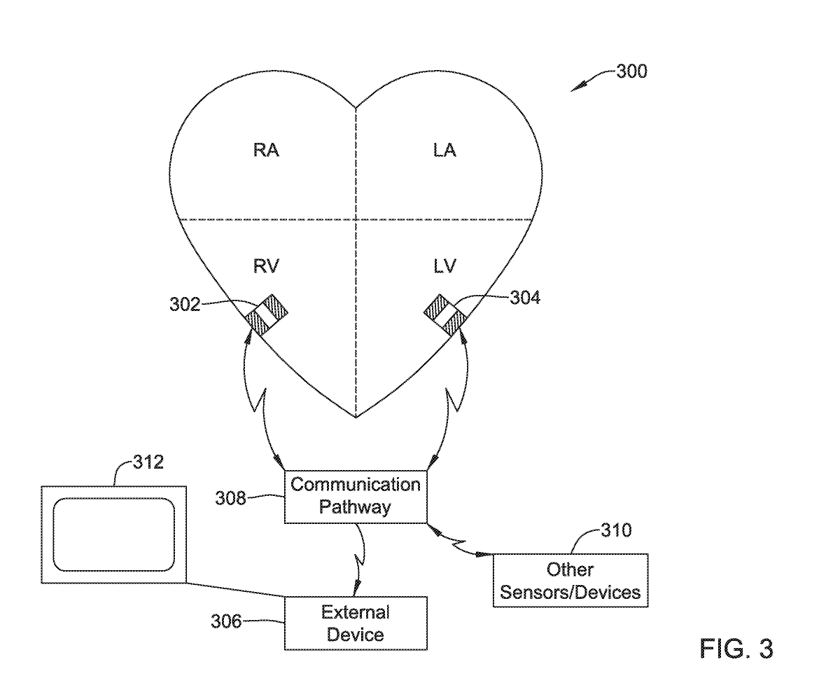

FIG. 3 shows an example medical device system with a communication pathway through which multiple medical devices 302, 304, 306, and/or 310 may communicate. In the example shown, the medical device system 300 may include LCPs 302 and 304, an external medical device 306, and other sensors/devices 310. The external device 306 may be any of the devices described previously with respect to MD 200. In some embodiments, the external device 306 may be provided with or be in communication with a display 312. The display 312 may be a personal computer, tablet computer, smart phone, laptop computer, or other display as desired. In some instances, the display 312 may include input means for receiving an input from a user. For example, the display 312 may also include a keyboard, mouse, actuatable buttons, or a touchscreen display. These are just examples. The other sensors/devices 310 may be any of the devices described previously with respect to the MD 200. In some instances, the other sensors/devices 310 may include a sensor, such as an accelerometer or blood pressure sensor, or the like. In some cases, the other sensors/devices 310 may include an external programmer device that may be used to program one or more devices of the system 300.

Various devices of the system 300 may communicate via a communication pathway 308. For example, the LCPs 302 and/or 304 may sense intrinsic cardiac electrical signals and may communicate such signals to one or more other devices 302/304, 306, and 310 of the system 300 via the communication pathway 308. In one example, one or more of the devices 302/304 may receive such signals and, based on the received signals, determine an occurrence of an arrhythmia. In some cases, the device or devices 302/304 may communicate such determinations to one or more other devices 306 and 310 of the system 300. In some cases, one or more of the devices 302/304, 306, and 310 of the system 300 may take action based on the communicated determination of an arrhythmia, such as by delivering a suitable electrical stimulation to the heart of the patient. In another example, the LCPs 302 and/or 304 may sense indications of blood pressure (e.g. via one or more pressure sensors) and indications of volume (e.g. via an impedance between the electrodes of an LCP or between LCPs). In one example, one or more of the devices 302/304 may receive such signals and, based on the received signals, determine a pressure-volume loop, and in some cases may communicate such information to one or more other devices 302/304, 306, and 310 of the system 300 via the communication pathway 308.

It is contemplated that the communication pathway 308 may communicate using RF signals, inductive coupling, optical signals, acoustic signals, conducted communication signals, and/or any other signals suitable for communication. Additionally, in at least some examples, the device communication pathway 308 may comprise multiple signal types. For instance, the other sensors/device 310 may communicate with the external device 306 using a first signal type (e.g. RF communication) but communicate with the LCPs 302/304 using a second signal type (e.g. conducted communication). Further, in some examples, communication between devices may be limited. For instance, as described above, in some examples, the LCPs 302/304 may communicate with the external device 306 only through the other sensors/devices 310, where the LCPs 302/304 send signals to the other sensors/devices 310, and the other sensors/devices 310 relay the received signals to the external device 306.

In some cases, the communication pathway 308 may include conducted communication. Accordingly, devices of the system 300 may have components that allow for such conducted communication. For instance, the devices of the system 300 may be configured to transmit conducted communication signals (e.g. current and/or voltage pulses) into the patient's body via one or more electrodes of a transmitting device, and may receive the conducted communication signals (e.g. pulses) via one or more electrodes of a receiving device. The patient's body may “conduct” the conducted communication signals (e.g. pulses) from the one or more electrodes of the transmitting device to the electrodes of the receiving device in the system 300. In such examples, the delivered conducted communication signals (e.g. pulses) may differ from pacing or other therapy signals. For example, the devices of the system 300 may deliver electrical communication pulses at an amplitude/pulse width that is sub-threshold to the heart. Although, in some cases, the amplitude/pulse width of the delivered electrical communication pulses may be above the capture threshold of the heart, but may be delivered during a refractory period of the heart and/or may be incorporated in or modulated onto a pacing pulse, if desired.

Delivered electrical communication pulses may be modulated in any suitable manner to encode communicated information. In some cases, the communication pulses may be pulse width modulated or amplitude modulated. Alternatively, or in addition, the time between pulses may be modulated to encode desired information. In some cases, conducted communication pulses may be voltage pulses, current pulses, biphasic voltage pulses, biphasic current pulses, or any other suitable electrical pulse as desired.

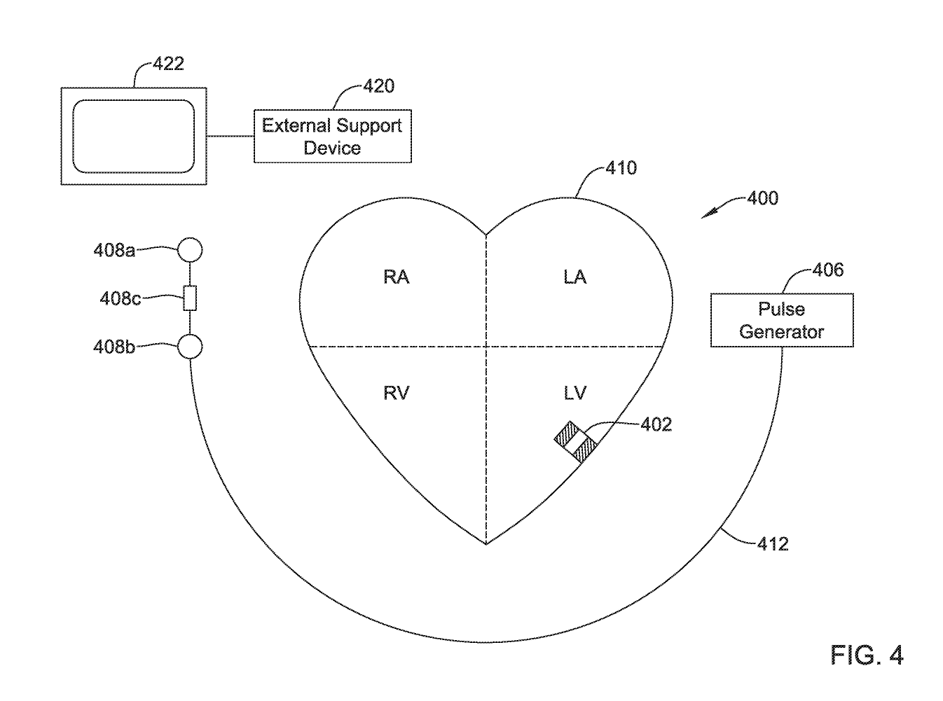

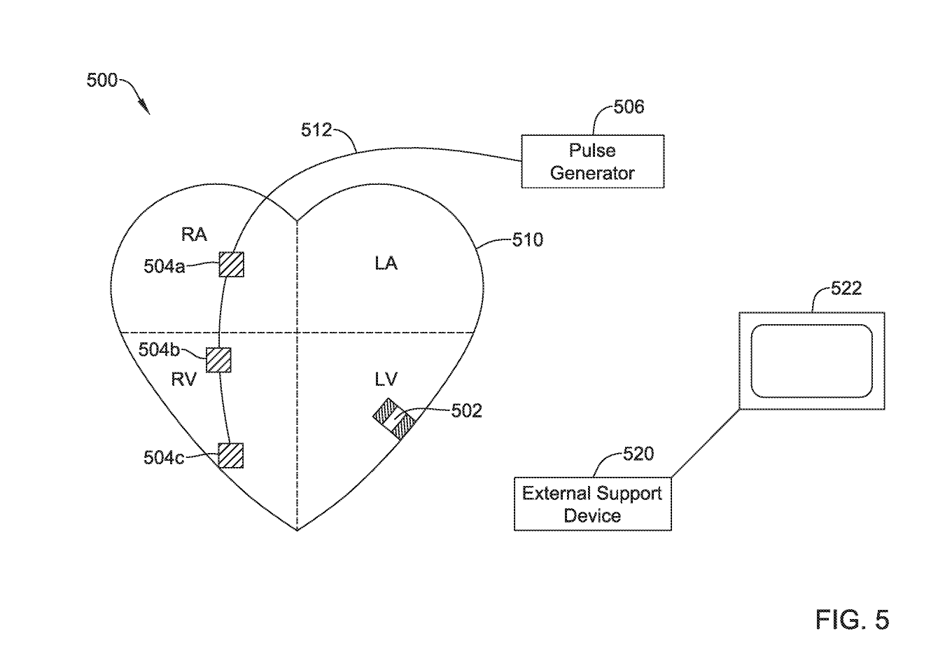

FIGS. 4 and 5 show illustrative medical device systems that may be configured to operate according to techniques disclosed herein. In FIG. 4, an LCP 402 is shown fixed to the interior of the left ventricle of the heart 410, and a pulse generator 406 is shown coupled to a lead 412 having one or more electrodes 408a, 408b, 408c. In some cases, the pulse generator 406 may be part of a subcutaneous implantable cardioverter-defibrillator (S-ICD), and the one or more electrodes 408a, 408b, 408c may be positioned subcutaneously adjacent the heart. In some cases, the S-ICD lead may extend subcutaneously from the S-ICD can, around the sternum and one or more electrodes 408a, 408b, 408c may be positioned adjacent the interior surface of the sternum. In some cases, the LCP 402 may communicate with the subcutaneous implantable cardioverter-defibrillator (S-ICD).

In some cases, the LCP 402 may be in the right ventricle, right atrium or left atrium of the heart, as desired. In some cases, more than one LCP 402 may be implanted. For example, one LCP may be implanted in the right ventricle and another may be implanted in the right atrium. In another example, one LCP may be implanted in the right ventricle and another may be implanted in the left ventricle. In yet another example, one LCP may be implanted in each of the chambers of the heart.

In FIG. 5, an LCP 502 is shown fixed to the interior of the left ventricle of the heart 510, and a pulse generator 506 is shown coupled to a lead 512 having one or more electrodes 504a, 504b, 504c. In some cases, the pulse generator 506 may be part of an implantable cardiac pacemaker (ICP) and/or an implantable cardioverter-defibrillator (ICD), and the one or more electrodes 504a, 504b, 504c may be positioned in the heart 510. In some cases, the LCP 502 may communicate with the implantable cardiac pacemaker (ICP) and/or an implantable cardioverter-defibrillator (ICD).

The medical device systems 400 and 500 may include an external support device, such as external support devices 420 and 520. The external support devices 420 and 520 can be used to perform functions such as device identification, device programming and/or transfer of real-time and/or stored data between devices using one or more of the communication techniques described herein. As one example, communication between the external support device 420 and the pulse generator 406 is performed via a wireless communication mode, and communication between the pulse generator 406 and the LCP 402 is performed via a conducted communication mode. In some examples, communication between the LCP 402 and the external support device 420 is accomplished by sending communication information through the pulse generator 406. However, in other examples, communication between the LCP 402 and the external support device 420 may be via a communication module. In some embodiments, the external support devices 420, 520 may be provided with or be in communication with a display 422, 522. The display 422, 522 may be a personal computer, tablet computer, smart phone, laptop computer, or other display as desired. In some instances, the display 422, 522 may include input means for receiving an input from a user. For example, the display 422, 522 may also include a keyboard, mouse, actuatable buttons, or be a touchscreen display. These are just examples.

FIGS. 4-5 illustrate two examples of medical device systems that may be configured to operate according to techniques disclosed herein. Other example medical device systems may include additional or different medical devices and/or configurations. For instance, other medical device systems that are suitable to operate according to techniques disclosed herein may include more or less LCPs implanted within the heart. Another example medical device system may include a plurality of LCPs without other devices such as the pulse generator 406 or 506, with at least one LCP capable of delivering defibrillation therapy. In yet other examples, the configuration or placement of the medical devices, leads, and/or electrodes may be different from those depicted in FIGS. 4 and 5. Accordingly, it should be recognized that numerous other medical device systems, different from those depicted in FIGS. 4 and 5, may be operated in accordance with techniques disclosed herein. As such, the examples shown in FIGS. 4 and 5 should not be viewed as limiting in any way.

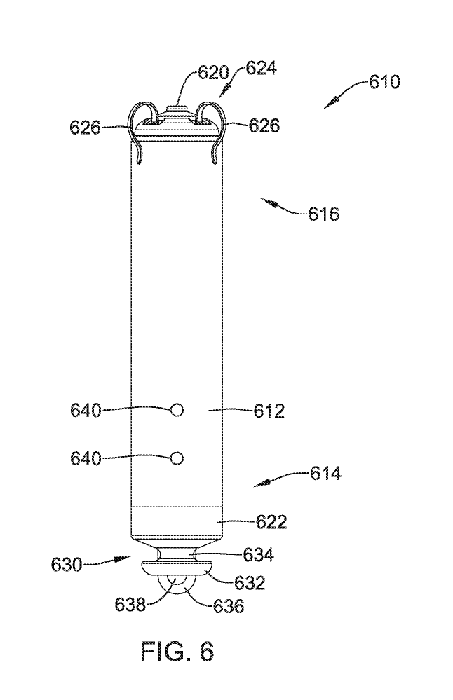

FIG. 6 is a side view of an illustrative implantable medical device. In the example shown, the implantable medical device is a leadless cardiac pacemaker (LCP) 610. The LCP 610 may be similar in form and function to the LCP 100 described above. The LCP 610 may include any of the modules and/or structural features described above with respect to the LCP 100 described above. The LCP 610 may include a shell or housing 612 having a proximal end 614 and a distal end 616. The illustrative LCP 610 includes a first electrode 620 secured relative to the housing 612 and positioned adjacent to the distal end 616 of the housing 612 and a second electrode 622 secured relative to the housing 612 and positioned adjacent to the proximal end 614 of the housing 612. In some cases, the housing 612 may include a conductive material and may be insulated along a portion of its length. A section along the proximal end 614 may be free of insulation so as to define the second electrode 622. The electrodes 620, 622 may be sensing and/or pacing electrodes to provide electro-therapy and/or sensing capabilities. The first electrode 620 (e.g. cathode) may be capable of being positioned against or may otherwise contact the cardiac tissue of the heart while the second electrode 622 (e.g. anode) may be spaced away from the first electrode 620. The first and/or second electrodes 620, 622 may be exposed to the environment outside the housing 612 (e.g. to blood and/or tissue).

In some cases, the LCP 610 may include a pulse generator (e.g., electrical circuitry) and a power source (e.g., a battery) within the housing 612 to provide electrical signals to the electrodes 620, 622 to control the pacing/sensing electrodes 620, 622. While not explicitly shown, the LCP 610 may also include, a communications module, an electrical sensing module, a mechanical sensing module, and/or a processing module, and the associated circuitry, similar in form and function to the modules 102, 106, 108, 110 described above. The various modules and electrical circuitry may be disposed within the housing 612. Electrical communication between the pulse generator and the electrodes 620, 622 may provide electrical stimulation to heart tissue and/or sense a physiological condition.

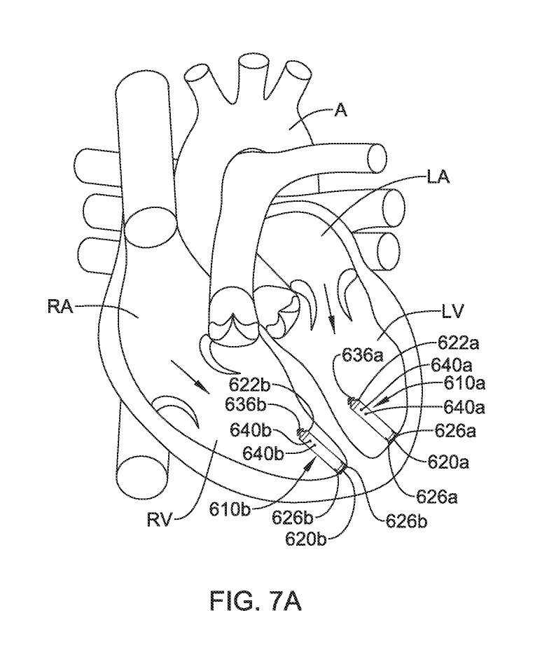

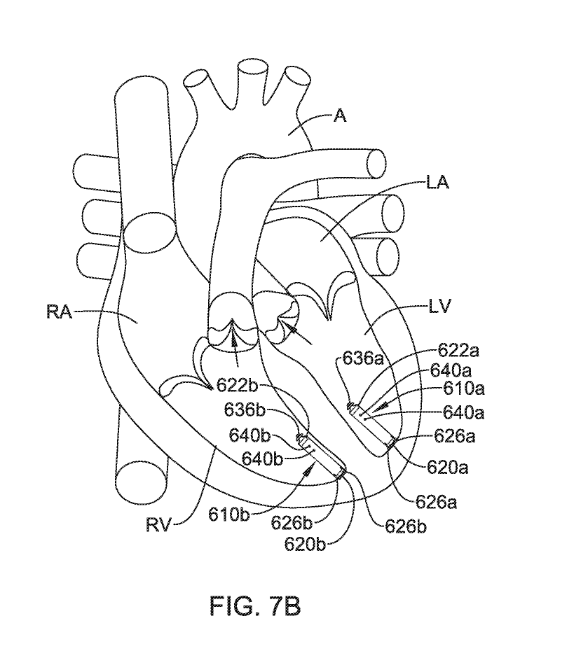

In the example shown, the LCP 610 includes a fixation mechanism 624 proximate the distal end 616 of the housing 612. The fixation mechanism 624 is configured to attach the LCP 610 to a wall of the heart H, or otherwise anchor the LCP 610 to the anatomy of the patient. As shown in FIGS. 6, 7A and 7B, in some instances, the fixation mechanism 624 may include one or more, or a plurality of hooks or tines 626 anchored into the cardiac tissue of the heart H to attach the LCP 610 to a tissue wall. In other instances, the fixation mechanism 624 may include one or more, or a plurality of passive tines, configured to entangle with trabeculae within the chamber of the heart H and/or a helical fixation anchor configured to be screwed into a tissue wall to anchor the LCP 610 to the heart H. These are just examples.

The LCP 610 may further include a docking member 630 proximate the proximal end 614 of the housing 612. The docking member 630 may be configured to facilitate delivery and/or retrieval of the LCP 610. For example, the docking member 630 may extend from the proximal end 614 of the housing 612 along a longitudinal axis of the housing 612. The docking member 630 may include a head portion 632 and a neck portion 634 extending between the housing 612 and the head portion 632. The head portion 632 may be an enlarged portion relative to the neck portion 634. For example, the head portion 632 may have a radial dimension from the longitudinal axis of the LCP 610 that is greater than a radial dimension of the neck portion 634 from the longitudinal axis of the LCP 610. In some cases, the docking member 630 may further include a tether retention structure 636 extending from or recessed within the head portion 632. The tether retention structure 636 may define an opening 638 configured to receive a tether or other anchoring mechanism therethrough. While the retention structure 636 is shown as having a generally “U-shaped” configuration, the retention structure 636 may take any shape that provides an enclosed perimeter surrounding the opening 638 such that a tether may be securably and releasably passed (e.g. looped) through the opening 638. In some cases, the retention structure 636 may extend though the head portion 632, along the neck portion 634, and to or into the proximal end 614 of the housing 612. The docking member 630 may be configured to facilitate delivery of the LCP 610 to the intracardiac site and/or retrieval of the LCP 610 from the intracardiac site. While this describes one example docking member 630, it is contemplated that the docking member 630, when provided, can have any suitable configuration.

It is contemplated that the LCP 610 may include one or more pressure sensors 640 coupled to or formed within the housing 612 such that the pressure sensor(s) is exposed to and/or otherwise operationally coupled with the pressure in the environment outside the housing 612. In some cases, the pressure sensor 640 may be coupled to an exterior surface of the housing 612. In other cases, the pressures sensor 640 may be positioned within the housing 612 with a pressure acting on the housing and/or a port on the housing 612 to affect the pressure sensor 640. The one or more pressure sensors 640 may be used to measure blood pressure within the heart. For example, if the LCP 610 is placed in the left ventricle, the pressure sensor(s) 640 may measure the pressure within the left ventricle. If the LCP 610 is placed in another portion of the heart (such as one of the atriums or the right ventricle), the pressures sensor(s) may measure the pressure within that portion of the heart. The pressure sensor(s) 640 may include a MEMS device, such as a MEMS device with a pressure diaphragm and piezoresistors on the diaphragm, a piezoelectric sensor, a capacitor-Micro-machined Ultrasonic Transducer (cMUT), a condenser, a micromanometer, or any other suitable sensor adapted for measuring cardiac pressure. The pressures sensor(s) 640 may be part of a mechanical sensing module described herein. It is contemplated that the pressure measurements obtained from the pressures sensor(s) 640 may be used to generate a pressure curve over cardiac cycles. The pressure readings may be taken in combination with impedance measurements (e.g. the impedance between electrodes 620 and 622, or other electrodes) to generate a pressure-impedance loop for one or more cardiac cycles, although this is not required. The impedance may be a surrogate for chamber volume, and thus the pressure-impedance loop may be representative of a pressure-volume loop for the heart H.

In some embodiments, the LCP 610 may be configured to measure impedance between the electrodes 620, 622. More generally, the impedance may be measured between any suitable electrode pair, which may include one or more of the additional electrodes 114′ described above. In some cases, the impedance may be measured between two spaced LCP's, such as two LCP's implanted within the same chamber (e.g. LV) of the heart H, or two LCP's implanted in different chambers of the heart H (e.g. RV and LV).

The processing module of the LCP 610 and/or external support devices may derive a measure of cardiac volume from intracardiac impedance measurements made between the electrodes 620, 622 (or other electrodes). Primarily due to the difference in the resistivity of blood and the resistivity of the cardiac tissue of the heart H, the impedance measurement may vary during a cardiac cycle as the volume of blood (and thus the volume of the chamber) surrounding the LCP changes. In some cases, the measure of cardiac volume may be a relative measure, rather than an actual measure. In some cases, the intracardiac impedance may be correlated to an actual measure of cardiac volume via a calibration process, sometimes performed during implantation of the LCP(s). During the calibration process, the actual cardiac volume may be determined using fluoroscopy, ultrasound, or the like, and the measured impedance may be correlated to the actual cardiac volume. In general, a lower impedance may be associated with a larger cardiac volume while a higher impedance may be associated with a smaller cardiac volume.

In some cases, the LCP 610 may include energy delivery circuitry operatively coupled to the first electrode 620 and the second electrode 622 for causing a current to flow between the first electrode 620 and the second electrode 622 in order to determine the impedance between the two electrodes 620, 622 (or other electrode pair). It is contemplated that the energy delivery circuitry may also be configured to deliver pacing pulses via the first and/or second electrodes 620, 622 (or other electrode pair). The LCP 610 may further include detection circuitry operatively coupled to the first electrode 620 and the second electrode 622 (or preferably another electrode pair when current is provided between the first electrode 620 and the second electrode 622) for detecting an electrical signal received between the first electrode 620 and the second electrode 622 (or preferably between the other electrode pair). In some instances, the detection circuitry may be configured to detect cardiac signals received between the first electrode 620 and the second electrode 622.

When the energy delivery circuitry delivers a current between the first electrode 620 and the second electrode 622, the detection circuitry may measure a resulting voltage between the first electrode 620 and the second electrode 622 (or preferably between a third and fourth electrode separate from the first electrode 620 and the second electrode 622) to determine the impedance. When the energy delivery circuitry delivers a voltage between the first electrode 620 and the second electrode 622, the detection circuitry may measure a resulting current between the first electrode 620 and the second electrode 622 (or between a third and fourth electrode separate from the first electrode 620 and the second electrode 622) to determine the impedance.

In some embodiments, the impedance may be measured between electrodes on different devices and/or in different heart chambers. For example, impedance may be measured between a first electrode in the left ventricle and a second electrode in the right ventricle. In another example, impedance may be measured between a first electrode of a first LCP in the left ventricle and a second LCP in the left ventricle. In yet another example, impedance may be measured from a current injected from outside of the heart. For example, a medical device (such as, but not limited to an S-ICD), may inject a known current into the heart and the LCP implanted in the heart H may measure a voltage resulting from the injected current to determine the impedance. These are just some examples.

FIG. 7A is a plan view of the example leadless cardiac pacing device 610a implanted within a left ventricle LV and an example leadless cardiac pacing device 610b implanted within a right ventricle RV of the heart H during ventricular filling. While two leadless cardiac pacemakers are used in this example, it is contemplated that the devices 610a and/or 610b could be merely diagnostic devices or any other suitable medical devices, as desired. The right atrium RA, left atrium LA, and aorta A are also illustrated. As used herein, reference to a leadless cardiac pacing device or leadless cardiac pacing device structure may lack the “a” or “b” denotation while the “a” and “b” denotation may be used to differentiate between two leadless cardiac pacing device within a heart. Further, it should be understood that the same medical device may not necessarily be used in each ventricle. FIG. 7B is a plan view of leadless cardiac pacing devices 610a, 610b (collectively 610) implanted within each of a left ventricle LV and a right ventricle RV of the heart H during a ventricular contraction. These figures illustrate how the volume of the left ventricle may change over a cardiac cycle. As can be seen in FIGS. 7A and 7B, the volume of the left ventricle during ventricular filling is larger than the volume of the left ventricle of the heart during ventricular contraction.

At each of a plurality of times during a cardiac cycle, the LCP 610a and LCP 610b may each be configured to capture a pressure via the corresponding pressure sensors 640a and 640b in the respective LV and RV chambers, resulting in a plurality of pressure-pressure data pairs for each cardiac cycle. In some cases, the pressure-pressure data pairs may be averaged over a plurality of cardiac cycles, if desired. Such pressure-pressure data pairs may be used to generate a ventricular pressure-pressure loop. In some cases, one or more parameters may be extracted or derived from the ventricle pressure-pressure loop. In any event, the ventricle pressure-pressure loop may facilitate cardiac resynchronization therapy (CRT), patient health status monitoring, and/or the management of a non-CRT cardiac therapy.

Alternatively, or additionally, at each of a plurality of times during a cardiac cycle, the LCP 610a and LCP 610b may each be configured to determine an impedance between their first and second electrodes in the respective LV and RV chambers, resulting in a plurality of impedance-impedance data pairs for each cardiac cycle. In some cases, the impedance-impedance data pairs may be averaged over a plurality of cardiac cycles, if desired. Such impedance-impedance data pairs may be used to generate a ventricular volume-volume loop. In some cases, one or more parameters may be extracted or derived from the ventricle volume-volume loop. In any event, the ventricle volume-volume loop may facilitate cardiac resynchronization therapy (CRT), patient health status monitoring, and/or the management of a non-CRT cardiac therapy.

In some embodiments, a corresponding pressure within the heart (e.g. left ventricle and/or right ventricle) may be captured at the same time as the corresponding impedance measurement, resulting in a plurality of pressure-impedance data pairs for each cardiac cycle. In some cases, the pressure-impedance data pairs may be averaged over a plurality of cardiac cycles, if desired. Such pressure-impedance data pairs may be used to generate a ventricular pressure-volume loop. In some cases, one or more parameters may be extracted or derived from the ventricle pressure-volume loop. In any event, the ventricle pressure-volume loop may facilitate cardiac resynchronization therapy (CRT), patient health status monitoring, and/or the management of a non-CRT cardiac therapy.

LCP 610a and LCP 610b may be synchronized in the taking of the pressure and/or impedance information to produce pressure-pressure pairs and/or impedance-impedance pairs that are aligned in time. In some cases, the LCP 610a and LCP 610b may receive a synchronization marker from a remote device. In some cases, the LCP 610a sends a synchronization marker to the LCP 610b. Each data pair may be associated with a corresponding time, and the time may be expressed relative to the synchronization maker. This is one example synchronization scheme. It is contemplated that any suitable synchronization scheme may be used as desired. In other examples, synchronization can be achieved by pace pulse detection of LCP 610a by LCP 610b (or vice versa) with pace timing information communicated from LCP 610a to LCP 610b (or vice versa) or via a third device.

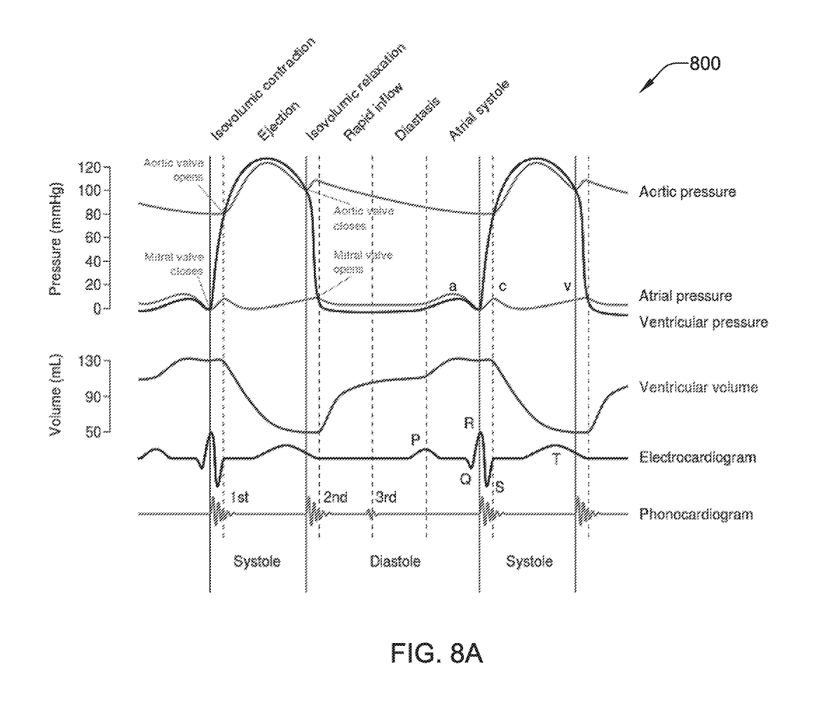

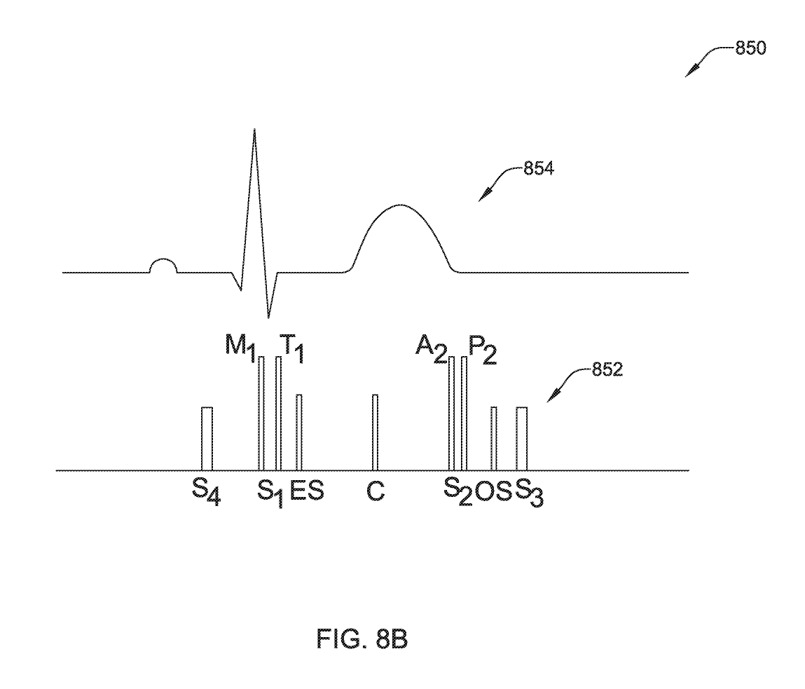

FIG. 8A is a graph 800 showing example pressures and volumes within a heart over time. More specifically, FIG. 8A depicts the aortic pressure, left ventricular pressure, left atrial pressure, left ventricular volume, an electrocardiogram (ECG or egram), and heart sounds of the heart H. A cardiac cycle may begin with diastole, and the mitral valve opens. The ventricular pressure falls below the atrial pressure, resulting in the ventricular filling with blood. During ventricular filling, the aortic pressure slowly decreases as shown. During systole, the ventricle contracts. When ventricular pressure exceeds the atrial pressure, the mitral valve closes, generating the S1 heart sound. Before the aortic valve opens, an isovolumetric contraction phase occurs where the ventricle pressure rapidly increases but the ventricle volume does not significantly change. Once the ventricular pressure equals the aortic pressure, the aortic valve opens and the ejection phase begins where blood is ejected from the left ventricle into the aorta. The ejection phase continues until the ventricular pressure falls below the aortic pressure, at which point the aortic valve closes, generating the S2 heart sound. At this point, the isovolumetric relaxation phase begins and ventricular pressure falls rapidly until it is exceeded by the atrial pressure, at which point the mitral valve opens and the cycle repeats. Cardiac pressure curves for the pulmonary artery, the right atrium, and the right ventricle, and the cardiac volume curve for the right ventricle, similar to those illustrated in FIG. 8A for the left part of the heart, may be likewise generated. Typically, the cardiac pressure in the right ventricle is lower than the cardiac pressure in the left ventricle.

In one example, the heart sound signals can be recorded using acoustic sensors, (for example, a microphone), which capture the acoustic waves resulted from heart sounds. In another example, the heart sound signals can be recorded using accelerometers or pressure sensors that capture the accelerations or pressure waves caused by heart sounds. The heart sound signals can be recorded within or outside the heart. These are just examples.

Ventricular dyssynchrony is a difference in the timing of the contraction of the left and right ventricles in the heart. In other words, it represents a lack of synchrony between the two ventricles. Large differences in the timing of the contraction of the ventricles may reduce cardiac efficiency and may be associated with heart failure. It is contemplated that the pressure of each of the left and right ventricles over one or more cardiac cycles may be used to identify and treat such ventricular dyssynchrony.

FIG. 8B is a graph 850 illustrating heart sounds 852 relative to an electrocardiogram 854. The two major heart sounds that can be heard are termed S₁ and S₂. The S₁ heart sound occurs at the beginning of ventricular systole and corresponds to the closure of the atrioventricular valves. In some instances, the S₁ heart sound can be heard as two components M₁ and T₁, corresponding to the closure of the mitral and tricuspid valves, respectively. The S₂ heart sound corresponds to the closure of the semilunar valves. In some instances, the S₂ heart sound can be heard as two components A₂ and P₂, corresponding to the closure of the aortic and pulmonic valves, respectively. A fourth heart sound S₄ precedes the S₁ heart sound. A pulmonic or aortic ejection sound ES may occur shortly after the S₁ heart sound. If present, the systolic click C of mitral valve prolapse may be heard in mid or late systole. The opening snap OS of mitral stenosis, if present, may be heard after the S₂ heart sound. A third heart sound S₃ can be heard after the S₂ heart sound.

It is contemplated that the heart sounds may be used to identify and/or quantify the degree of ventricular-ventricular dyssynchrony. When V-V synchrony worsens (e.g. becomes more dyssynchronous), the two components M₁ and T₁ of the S₁ heart sound may become further apart from each other, resulting in a split of the S₁ or a wider S₁ heart sound. Similarly, the two components A₂ and P₂ of the S₂ heart sound may also become more separate (e.g. further apart) when V-V synchrony worsens.

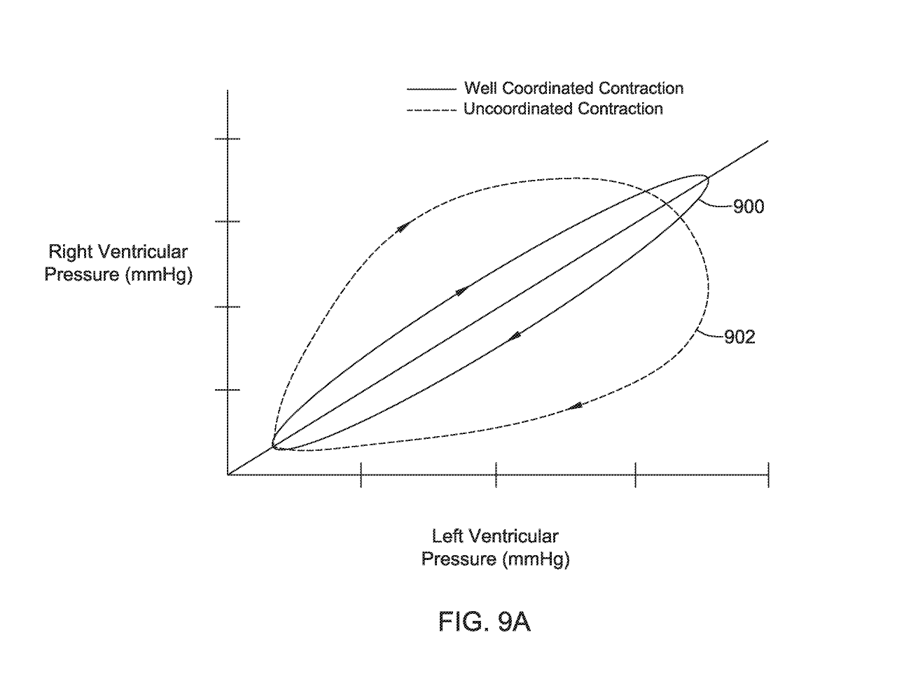

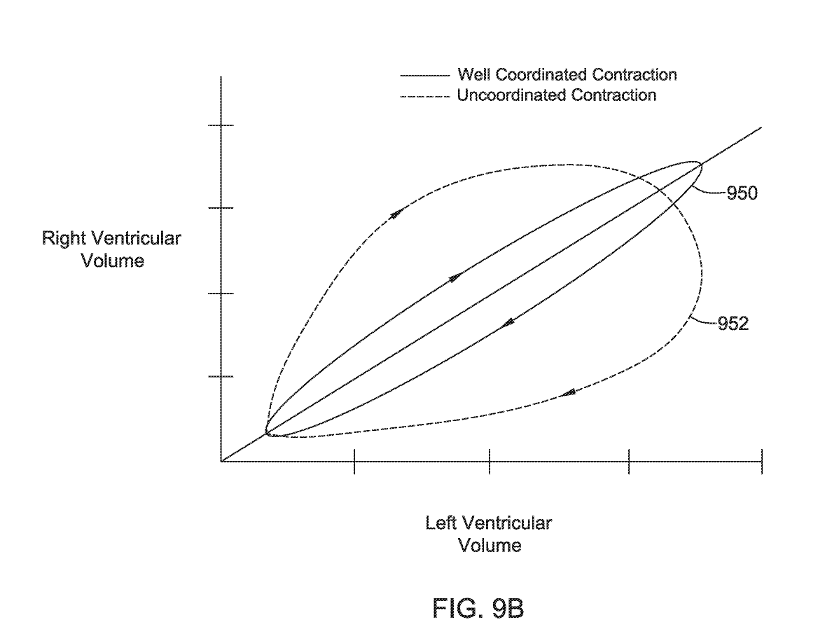

FIG. 9A illustrates a method of representing pressure parameters of the heart in a pressure-pressure (PP) loop. In some instances, the PP loop may be representative of the pressures in the left and right ventricles. PP loops can be used to determine performance characteristics of the heart. An illustrative PP loop 900 represents a heart where the right ventricle and the left ventricle are functioning in well-coordinated contractions (e.g. in synchrony) or, in other words, are nearly in phase. This results in a narrow PP loop as shown. In contrast, illustrative PP loop 902 shows a heart where the right ventricle is leading, or contracting before the left ventricle, resulting in a fatter PP loop as shown.

FIG. 9B illustrates a method of representing pressure parameters of the heart in a volume-volume (VV) loop. In some instances, the VV loop may be representative of the volumes in the left and right ventricles. VV loops can be used to determine performance characteristics of the heart. An illustrative VV loop 950 represents a heart where the right ventricle and the left ventricle are functioning in well-coordinated contractions (e.g. in synchrony) or, in other words, are nearly in phase. This results in a narrow VV loop as shown. In contrast, illustrative VV loop 952 shows a heart where the right ventricle is leading, or contracting before the left ventricle, resulting in a fatter VV loop as shown.

As can be seen in FIG. 9A, a pressure-pressure loop can provide a clinician with information regarding the heart's function. It is contemplated that a volume-volume loop may also provide a clinician with information regarding the heart's function. As such, and as described above, it may be desirable to capture and record data relating to the pressure and volume of various chambers of the heart over time. For example, the processing module and/or other control circuitry of any of the above described LCP devices 100, 302, 304, 402, 502, 610 may be configured to determine a new pressure-pressure loop, a new volume-volume loop and/or a new pressure-volume loop every day, week, month, or during a doctor visit. The previous pressure-pressure loop, volume-volume loop, and/or pressure-volume loop may be stored for later use, such as to identify a change and/or trend. In some cases, the LCP device may store the pressure-pressure loops, volume-volume loops, and/or pressure-volume loops. In some cases, the LCP may transmit the pressure-pressure loops, volume-volume loops, and/or pressure-volume loops to another device, such as an S-ICD device or a remote device, to store the pressure-pressure loops, volume-volume loops, and/or pressure-volume loops. In some cases, the LCP may transmit the pressure-pressure loop data pairs, volume-volume loop data pairs, and/or pressure-volume loop data pairs to another device, such as an S-ICD device or a remote device, to store the pressure-pressure loop data pairs, volume-volume loop data pairs, and/or pressure-volume loop data pairs. The pressure-pressure loop(s), volume-volume loop(s), and/or pressure-volume loop(s) may facilitate cardiac rhythm therapy (CRT), patient health status monitoring, and/or the management of a non-CRT cardiac therapy. While two LCPs may be used to collect the pressure and/or impedance pairs as described above, it is contemplated that a diagnostic device may be used that has no pacing capabilities. The diagnostic device may include a pressure sensor and/or two or more electrodes for measuring an impedance and/or for communicating with an LCP, S-ICD, or other implanted or external device. The diagnostic device may be implanted with a chamber of the heart H. In some cases, a diagnostic device and LCP, both implanted in the same or different chamber, may cooperate to collect the desired pressure pairs, impedance pairs, and/or pressure-impedance pairs. For example, current and/or voltage may be delivered between a first electrode on a diagnostic device and a second electrode on an LCP, or vice versa to collect the impedance (e.g. volume) information. Alternatively, or additionally, the diagnostic device may collect pressure information while the LCP may collect impedance information.

In some embodiments, PP and/or VV loops (e.g. see, for example, FIGS. 9A and 9B) are created and presented to a user via a user interface. The user may use the displayed PP and/or VV loops to determine a patient condition or understand how another portion of the system (e.g. an algorithm residing in one or more LCPs) made a determination of a patient condition. In some embodiments, the PP and/or VV loop figures may not be displayed to a user but rather used only algorithmically by the system to determine a patient condition.

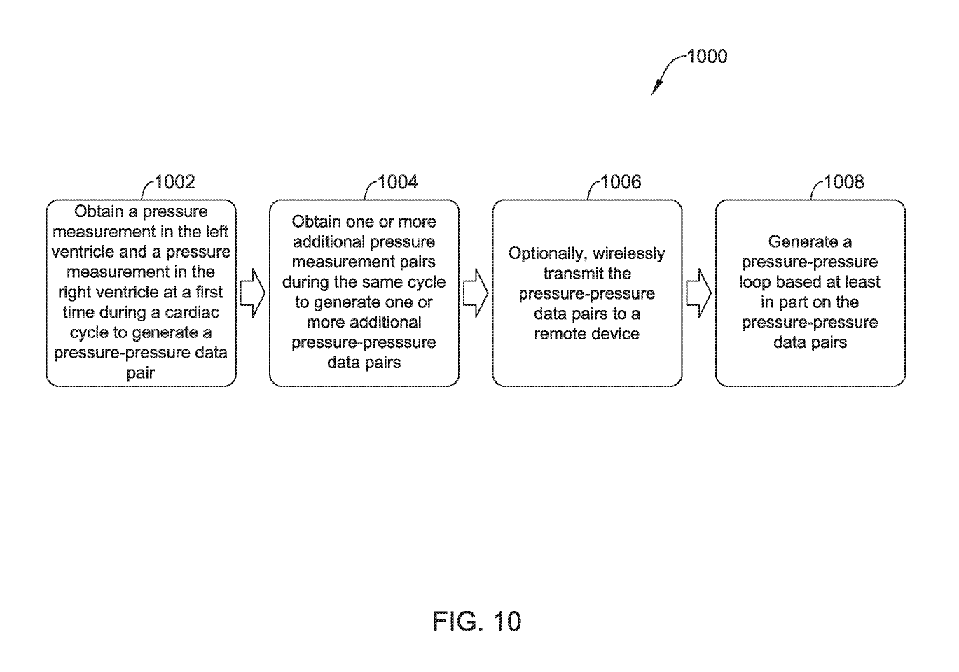

FIG. 10 is a flow chart of an illustrative method 1000 for generating a pressure-pressure loop from data obtained from a pair of implanted LCPs, such as any of the above described LCP devices 100, 302, 304, 402, 502, 610. While the method is described using an LCP in each ventricle, it is contemplated that other devices and/or combinations of devices may be used. For example, two or more LCPs may be used as a system to collect data from different chambers in the heart. In another example, two LCPs may be used in combination with an S-ICD device. In another example, a diagnostic device may be implanted with each chamber, and may be used to collect the data from different chambers in the heart. In addition to collecting data from the different chambers, the diagnostic device may also record contextual data. Contextual data may include, but is not limited to patient metabolic demands, activity level (e.g. active, inactive), posture (sitting, standing, lying down, etc.), sleep status, etc. The device and/or clinician may use the contextual data to facilitate therapy decision. For example, PP, PV, and/or VV loops may be inhibited when patients are in certain conditions, for example, but not limited to, exertion, recent exertion, unusual postures (e.g. yoga), or when patients are outside certain operating conditions, for example, but not limited to, lying down, seated, etc.

As described above, in the example shown, each LCP may include a processing module that includes control circuitry configured to control the operation of the LCP. In some instances, the processing module may include separate circuits for therapy delivery, hemodynamic (e.g. pressure) sensing, and/or volume sensing, although this is not required. It is contemplated the processing module may further include an additional circuit or algorithm generating a PP loop which may be configured to convert the pressure data obtained from the pressure sensors on each of the LCPs into a PP loop, but this is not required.

As shown at block 1002, the processing module and/or any other circuits or sub-circuits of one or more LCPs (and/or other implantable devices) may obtain a result of a pressure measurement in the left ventricle and the result of a pressure measurement in the right ventricle. The pressure measurements are simultaneously, or substantially simultaneously, made at a first time during a cardiac cycle, resulting in a first pressure-pressure data pair. In one example, a first LCP may be placed in the left ventricle and a second LCP may be placed in the right ventricle. The first and second LCP may obtain pressure data simultaneously, or substantially simultaneously, at a first time during a cardiac cycle, resulting in a first pressure-pressure data pair. In some instances, the first time may correspond with an S₁ heart sound, although this is not required. It is contemplated that the heart sounds may be detected with a heart sound sensor on one or both LCP devices. If so provided, a heart sound sensor may also be used to time a split of the S₁ and/or S₂ heart sounds (e.g. time between S₁ and/or S₂ heart sounds of adjacent cardiac cycles). Alternatively, or additionally, the heart sounds may be detected with the pressure sensor(s).

The pressure-pressure data pair may be stored in a memory of one or both of the LCPs or in a remote device. In some instances, the data pair may be stored in a table along with a corresponding time and/or date stamp. In some cases, the data pair may be transmitted to a remote device, such as another LCP, an S-ICD device, or an external device. The processing module of each device may then obtain one or more additional pressure-pressure measurement pairs at different times during the same cardiac cycle, as shown at block 1004. For example, a second pressure-pressure data pair may be determined at a second time, a third pressure-pressure data pair may be determined at a third time, a fourth pressure-pressure data pair may be determined at a fourth time, etc. In some instances, the second time may correspond with an S₂ heart sound, although this is not required. The one or more additional data pairs may be stored in the memory of the LCP and/or an external device. In some cases, the LCP may transmit the pressure-pressure data pairs to another device, such as an S-ICD device or an external device, as shown at block 1006.

It is contemplated that the processing module of each LCP may be configured to sample and produce pressure-pressure data pairs at set time intervals or to obtain a predetermined number of pressure-pressure data pairs per cardiac cycle. It is contemplated that increasing the frequency of sampling may result in a more accurate and/or higher resolution PP loop. However, frequent sampling may decrease the life of the battery of the LCP(s). The use of the terms “first time” and “second time” are not intended to chronologically limit the order the pressure-pressure data pairs are obtained. In some instances, a plurality of corresponding pressures may be determine via the pressure sensors at a plurality of times between the first time and the second time, resulting in a plurality of additional pressure-pressure data pairs.

Once the LCPs have gathered pressure-pressure data pairs over at least one cardiac cycle, the resulting data pairs may be converted into a PP loop, as shown at block 1008. For example, the LCP(s) or an external device may be configured to generate a PP loop that is based, at least in part, on the plurality of data pairs.

It is contemplated that data pairs obtained over a plurality (e.g. two or more, five or more, ten or more) of cardiac cycles may be averaged to generate the PP loop. For example, the LCP circuitry may be configured to record or sample pressure data in each ventricle at the same (or similar) time points in each in a series of cardiac cycles such that the first pressure-pressure data pair from a first cardiac cycle can be averaged with the corresponding first pressure-pressure data pair from any number of subsequent (or preceding) cardiac cycles. Averaging the data over a plurality of cardiac cycles may reduce the noise and provide a more robust representation of the PP loop.

In some instances, the processing module may include circuitry to convert the data pairs into a PP loop. In other instances, the LCP circuitry may be configured to wirelessly transmit the first impedance-pressure data pair (and/or any additional data pairs) to a remote or external device, such as, but not limited to, any of the medical or external devices described above, as shown at block 1006. The LCP(s) may communicate with the remote or external device via radiofrequency (RF) signals, inductive coupling, optical signals, acoustic signals, conducted communication signals, and/or any other signals suitable for communication.

It is contemplated that the PP loop and/or metrics extracted from the PP loop may be used to optimize therapies and/or provide the clinician with information regarding cardiac functionality. In an embodiment the area (e.g. the region inside the loop) of the pressure-pressure loop(s) or volume-volume loop(s) is used to determine the degree of mechanical synchronization of the left and right ventricles wherein a relatively large loop area indicates dyssynchrony and a relatively small loop area indicates synchrony. In an embodiment, the relative phase of left and right ventricular contraction and relaxation is determined by the direction of travel around the pressure-pressure loop(s) or volume-volume loop(s) wherein a clockwise direction indicates a right ventricular event (e.g. contraction) preceding the associated left ventricular event (e.g. contraction) and wherein a counterclockwise direction indicates a left ventricular event preceding the associated right ventricular event (this assumes the left ventricular pressure or volume is plotted on the horizontal axis and the right ventricular pressure or volume is plotted on the vertical axis.

In some cases, the duration from a pace or sense event to a minimum volume event may be one metric that may reflect contraction efficiency and can be monitored as a function of pace timing parameters. In another embodiment, the PP loop and/or extracted metrics may be used to change a resynchronization timing parameter (e.g. the RV-to-LV delay or the atrial to ventricular delay when pacing). For example, if the loop area indicates excessive dyssynchrony and the loop direction indicates right ventricular contraction occurring before left ventricular contraction the RV-to-LV delay might be reprogrammed to a negative value (e.g. −50 milliseconds) such that the left ventricle is paced before the right ventricle. Pacing the left ventricle before the right ventricle might resolve the latency of the left ventricular contraction compared to the right ventricular contraction. In other embodiments, the electrodes used for pacing and/or sensing, pacing timing, and/or optimization of CRT and non-CRT therapies, etc. might be changed. In some cases, it is contemplated that when the PP loop is generated within the processing module of the LCP, the processing module may be programmed to optimize CRT therapies with or without a clinician viewing the PP loop.

In some instances, the PP loop may be processed to extract a metric related to the ellipticity of the PP loop. For the purposes of optimizing therapy, a greater ellipticity of the PP loop may be desired. One way to measure ellipticity could be to compute a covariance matrix using the data pairs (xᵢ,yᵢ) that constitute the PP loop, apply a Karhunen Loeve Transform (KLT) to obtain the two eigenvectors (and corresponding eigenvalues) that represent the spread along the two uncorrelated dimensions. Greater ellipticity can be inferred by non-uniform spread along the two dimensions as quantified by the two eigenvalues. A perfect circular spread would result in equal eigenvalues. Thus the relative difference between two eigenvalues would be one way of quantifying the ventricle-ventricle dyssynchrony and maximizing the relative difference would be one way of guiding the therapy optimization.

In some instances, the PP loop may be processed to extract metrics indicative of the strength of projections of the data pairs (xᵢ,yᵢ) constituting the PP loop along the vector V₁=[1,1] relative to the strength of projections along the vector V₂=[1,−1]. Specifically,

$${P}_{1} = {\sum }_{i}^{ } {V}_{1} * {[ {x}_{i} , {y}_{i} ]}^{T}$$

$${P}_{2} = {\sum }_{i}^{ } {V}_{2} * {[ {x}_{i} , {y}_{i} ]}^{T}$$

A well synchronized contraction as a result of a well optimized therapy will yield a substantial projection along V₁ (i.e P₁) and very minimal projection along V₂ (i.e P₂). Thus ratio of V₂ to V₁ would be one way of quantifying the ventricle-ventricle dyssynchrony and minimizing the ratio would be one way of guiding the therapy optimization.

In some instances, the pressure-pressure data pairs may be transmitted from the LCP(s) to a remote device, such as an S-ICD device, an external device or other device. It is contemplated that the remote device may extract various metrics from the pressure-pressure data pairs, and communicate information back to the LCP(s) to improve CRT and/or non-CRT therapies delivered by the LCP(s).

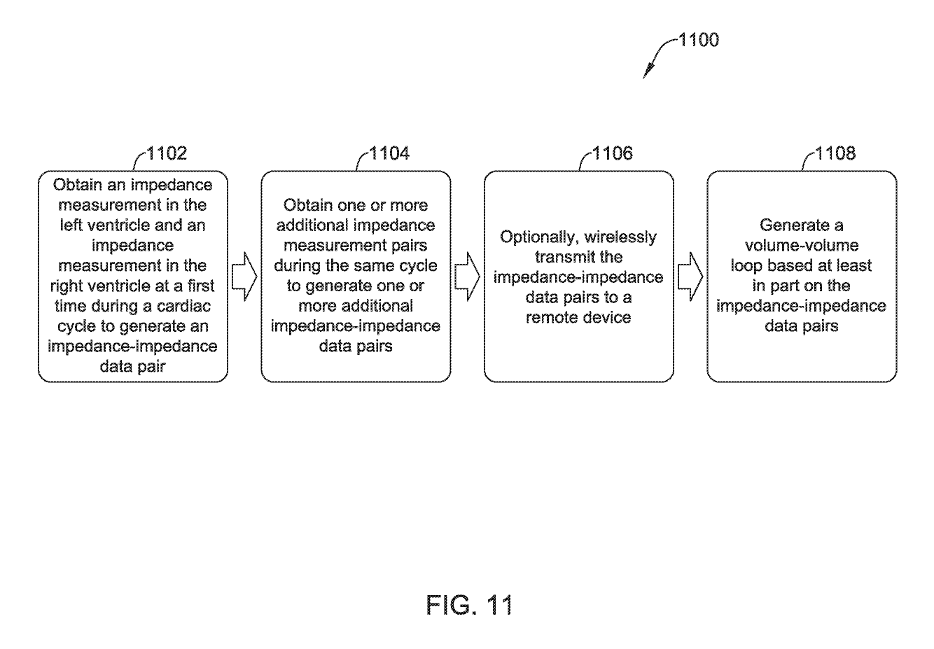

FIG. 11 is a flow chart of an illustrative method 1100 for generating a volume-volume loop from data obtained from a pair of implanted LCPs such as any of the above described LCP devices 100, 302, 304, 402, 502, 610. The VV loop may represent the volume of the right ventricle and the volume of the left ventricle over a cardiac cycle. The VV loop may be generated in place of or in addition to the PP loop described above. While the method is described using an LCP in each ventricle, it is contemplated that other devices and/or combinations of devices may be used. For example, two or more LCPs may be used as a system to collect data from different chambers in the heart. As described above, each LCP may include a processing module that includes control circuitry configured to control the operation of the LCP. In some instances, the processing module may include separate circuits for therapy delivery, hemodynamic (e.g. impedance) sensing, and/or volume sensing, although this is not required. It is contemplated the processing module may further include an additional circuit or algorithm generating a VV loop which may be configured to convert the impedance data obtained from the impedance measurements obtained from each of the LCPs into a VV loop.

As shown at block 1102, the processing module and/or any other circuits or sub-circuits of one or more LCPs (and/or other implantable devices) may obtain a result of an impedance measurement in the left ventricle and a result of an impedance measurement in the right ventricle simultaneously, or substantially simultaneously, at a first time during a cardiac cycle, resulting in a first impedance-impedance data pair. For example, a first LCP may be placed in the left ventricle and a second LCP may be placed in the right ventricle. The first and second LCP may obtain impedance data simultaneously, or substantially simultaneously, at a first time during a cardiac cycle, resulting in a first impedance-impedance data pair. In some instances, the first time may correspond with an S₁ heart sound, although this is not required. It is contemplated that the heart sounds may be detected with a heart sound sensor on one or both LCP devices. If so provided, a heart sound sensor may also be used to time a split of the S₁ and/or S₂ heart sounds (e.g. time between S₁ and/or S₂ heart sounds of adjacent cardiac cycles). Alternatively, or additionally, the heart sounds may be detected with the impedance sensor(s). The impedance-impedance data pair may be stored in a memory of one or both of the LCPs. In some instances, the data pair may be stored in a table along with a corresponding time and/or date stamp. In some cases, the data pair may be transmitted to a remote device, such as another LCP, an S-ICD device, or an external device.

The processing module of each device may then obtain one or more additional impedance-impedance measurement pairs at different times during the same cardiac cycle, as shown at block 1104. For example, a second impedance-impedance data pair may be determined at a second time, a third impedance-impedance data pair may be determined at a third time, a fourth impedance-impedance data pair may be determined at a fourth time, etc. In some instances, the second time may correspond with an S₂ heart sound, although this is not required. The one or more additional data pairs may be stored in the memory of the LCP. In some cases, the LCP may transmit the impedance-impedance data pairs to another device, such as an S-ICD device or an external device, as shown at block 1106. It is contemplated that the processing module may be configured to sample impedance-impedance data pairs at set time intervals or to obtain a predetermined number of impedance-impedance data pairs per cardiac cycle. It is contemplated that increasing the frequency of sampling may result in a more accurate and or higher resolution VV loop. However, frequent sampling may decrease the life of the battery of the LCP(s). The use of the terms “first time” and “second time” are not intended to chronologically limit the order the impedance-impedance data pairs are obtained.

Once the LCPs have gathered impedance-impedance data pairs over at least one cardiac cycle, the data pairs may be converted into a VV loop, as shown at block 1108. For example, the LCP(s) or an external device may be configured to generate a VV loop that is based, at least in part, on the plurality of data pairs. As described above, the impedance-impedance data pairs may be correlated to a volume of the ventricle(s) and may be converted into relative volume measurements. In some embodiments, the processing module and/or external device may be configured to generate an impedance-impedance (ZZ) loop instead of a VV loop. The ZZ loop may provide similar information as the VV loop but may require less processing. At a high level, the ZZ loop may be considered equivalent to the VV loop. However, it should be noted that a ZZ loop may be an inverted form of a VV loop with opposite rotation as impedance may be approximately inversely proportional to volume in each chamber.

It is contemplated that data pairs obtained over a plurality (e.g. two or more, five or more, ten or more) of cardiac cycles may be averaged to generate the VV loop. For example, the LCP circuitry may be configured to record or sample impedance data in each ventricle at the same (or similar) time points in each in a series of cardiac cycles such that the first impedance-impedance data pair from a first cardiac cycle can be averaged with the corresponding first impedance-impedance data pair from any number of subsequent (or preceding) cardiac cycles. Averaging the data over a plurality of cardiac cycles may reduce the noise and provide a more robust representation of the VV loop.

In some instances, the processing module may include circuitry to convert the data pairs into a VV loop. In other instances, the LCP circuitry may be configured to wirelessly transmit the first impedance-impedance data pair (and/or any additional data pairs) to a remote or external device, such as, but not limited to, any of the medical or external devices described above, as shown at block 1106. The LCP(s) may communicate with the remote or external device via radiofrequency (RF) signals, inductive coupling, optical signals, acoustic signals, conducted communication signals, and/or any other signals suitable for communication.

In some instances, the VV loop may be processed to extract a metric related to the ellipticity of the VV loop. For the purposes of optimizing therapy, a greater ellipticity of the VV loop may be desired. One way to measure ellipticity could be to compute a covariance matrix using the data pairs (xᵢ,yᵢ) that constitute the VV loop, apply a Karhunen Loeve Transform (KLT) to obtain the two eigenvectors (and corresponding eigenvalues) that represent the spread along the two uncorrelated dimensions. Greater ellipticity can be inferred by non-uniform spread along the two dimensions as quantified by the two eigenvalues. A perfect circular spread would result in equal eigenvalues. Thus the relative difference between two eigenvalues would be one way of quantifying the ventricle-ventricle dyssynchrony and maximizing the relative difference would be one way of guiding the therapy optimization.

In some instances, the VV loop may be processed to extract metrics indicative of the strength of projections of the data pairs (xᵢ,yᵢ) constituting the VV loop along the vector V₁=[1,1] relative to the strength of projections along the vector V₂=[1,−1]. Specifically,

$${P}_{1} = {\sum }_{i}^{ } {V}_{1} * {[ {x}_{i} , {y}_{i} ]}^{T}$$

$${P}_{2} = {\sum }_{i}^{ } {V}_{2} * {[ {x}_{i} , {y}_{i} ]}^{T}$$

A well synchronized contraction as a result of a well optimized therapy will yield a substantial projection along V₁ (i.e P₁) and very minimal projection along V₂ (i.e P₂). Thus ratio of V2 to V1 would be one way of quantifying the ventricle-ventricle to dyssynchrony and minimizing the ratio would be one way of guiding the therapy optimization.

In an embodiment the area (e.g. the region inside the loop) of the pressure-pressure loop(s) or volume-volume loop(s) is used to determine the degree of mechanical synchronization of the left and right ventricles wherein a relatively large loop area indicates dyssynchrony and a relatively small loop area indicates synchrony. In an embodiment the relative phase of left and right ventricular contraction and relaxation is determined by the direction of travel around the pressure-pressure loop(s) or volume-volume loop(s) wherein a clockwise direction indicates a right ventricular event (e.g. contraction) preceding the associated left ventricular event (e.g. contraction) and wherein a counterclockwise direction indicates a left ventricular event preceding the associated right ventricular event (this assumes the left ventricular pressure or volume is plotted on the horizontal axis and the right ventricular pressure or volume is plotted on the vertical axis.

In some cases, the duration from a pace or sense event to a minimum volume event may be one metric that may reflect contraction efficiency and can be monitored as a function of pace timing parameters. In another embodiment, the VV loop and/or extracted metrics may be used to change a resynchronization timing parameter (e.g. the RV-to-LV delay or the atrial to ventricular delay when pacing). For example, if the loop area indicates excessive dyssynchrony and the loop direction indicates right ventricular contraction occurring before left ventricular contraction the RV-to-LV delay might be reprogrammed to a negative value (e.g. −50 milliseconds) such that the left ventricle is paced before the right ventricle. Pacing the left ventricle before the right ventricle might resolve the latency of the left ventricular contraction compared to the right ventricular contraction. In other embodiments, the electrodes used for pacing and/or sensing, pacing timing, and/or optimization of CRT and non-CRT therapies, etc. might be changed. In some cases, it is contemplated that when the VV loop is generated within the processing module of the LCP, the processing module may be programmed to optimize CRT therapies with or without a clinician viewing the VV loop.

In some instances, the impedance-impedance data pairs may be transmitted from the LCP to a remote device, such as an S-ICD device, an external device or other device. It is contemplated that the remote device may extract various metrics from the impedance-impedance data pairs, and communicate information to the LCP to improve CRT and/or non-CRT therapies delivered by the LCP.

In some cases, it may be useful to store pressure and/or impedance measurements along with a corresponding time and/or date stamp from either or both of the LCPs. It is contemplated that the LCPs may receive a synchronization marker from a remote device that allows the times the pressure and/or impedance measurements are recorded to be expressed relative to the synchronization marker. In other words, a synchronization marker may be provided by a remote device to help insure the pressure and/or impedance measurements recorded at each device (e.g. an LCP in the left ventricle and an LCP in the right ventricle) are recorded at the same time or substantially the same time. This may reduce or eliminate the possibility of falsely reporting ventricular dyssynchrony due to unsynchronized data recording. Alternatively or additionally, one or both of the LCPs may provide the synchronization marker to each other or the remote device.

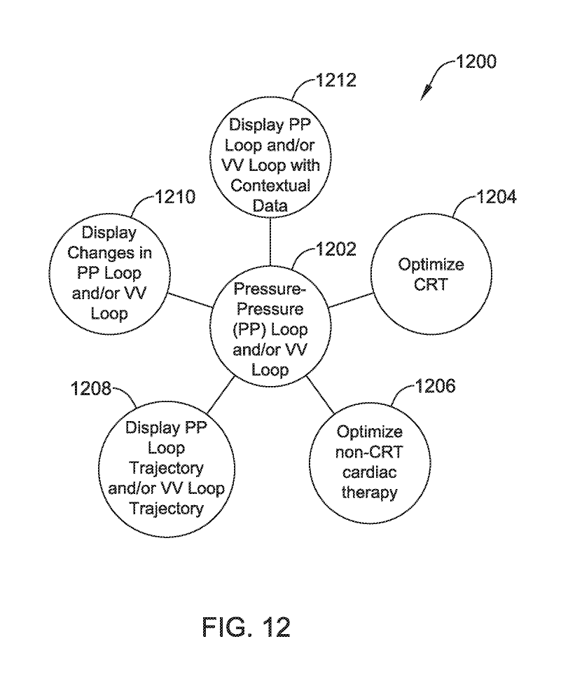

FIG. 12 is a diagram 1200 illustrating some, but not all, potential uses of one or more PP loops and/or VV loops 1202 sampled from one or more LCP's. The PP loop and/or VV loop 1202 may provide information that allows a clinician to optimize CRT timing 1204. For example, the edges, direction of travel, and area of a PP loop and/or VV loop 1202 may vary with the CRT timing. It may be desirable to minimize the area of a PP loop and/or VV loop 1202 and/or control a direction of travel to reduce ventricle-ventricle dyssynchrony. In some cases, these changes in the PP loop and/or VV loop 1202 may allow a clinician to identify proper CRT timing parameters. In some cases, these changes in the PP loop and/or VV loop 1202 may allow a clinician to identify pacing sites as not acceptable, acceptable, better, or best. In some cases the pacing site is identified during the implant procedure; in other cases the pacing site is identified after the implant procedure. In an implanted device with multiple electrodes (e.g. a multi-electrode LCP or multiple LCPs), multiple pacing sites may be available. The PP loop and/or VV loop 1202 may allow the clinician to identify and use the best pacing sites. Additionally, or alternatively, the PP loop and/or VV loop may also allow a clinician to optimize non-CRT cardiac therapy 1206 (e.g. effectiveness or dose of ACE inhibitors, diuretics, etc.). For example, PP loop and/or VV loop 1202 may allow a clinician to assess the impact of medications and/or understand how the heart failure status of a patient changes over time.

In some instances, multiple PP loops and/or VV loops generated from different cardiac cycles (or different average cardiac cycles) may be displayed simultaneously to display and/or illustrate a PP loop and/or VV loop trajectory or trend 1208 over time (e.g. minutes, days, weeks, months, years). In other words, the LCP and/or remote device may be configured to store and display a plurality of PP loops and/or VV loops generated over a period of time. The PP loop and/or VV loop trajectory 1208 may allow a clinician to view the effectiveness of a therapy (CRT and/or non-CRT). For example, a PP loop and/or VV loop trajectory 1208 may allow a clinician to see changes in the PP loop and/or VV loop after a drug change (dosage or type), a CRT timing change, an AV delay change, other therapy change, etc. APP loop and/or VV loop trajectory 1208 may also allow a clinician to determine or if there are changes not associated with a therapy change that might indicate changes in a current therapy or a new therapy are needed, such as decompensation of the heart H. The PP loop and/or VV loop trajectory 1208 may also be used to guide patient lifestyle changes.

Multiple PP loops and/or VV loops generated from different cardiac cycles (or different average cardiac cycles) may be compared to one another to determine and display changes in the PP loop and/or VV loop 1210. Some illustrative changes in the PP loop and/or VV loop may include, but are not limited to horizontal (e.g. width), vertical (e.g. height), area, slope, etc. It is further contemplated that a PP loop and/or VV loop 1202 may be displayed with contextual data 1212 indicating a patient's physiological state. Contextual data may include, but is not limited to patient metabolic demands, activity level (e.g. active, inactive), posture (sitting, standing, lying down, etc.), sleep status, a disease status, an effect of a therapy, a heart rate, a respiratory rate, a blood gas, a blood analyte, a drug therapy, etc. This data may be captured along with each of the pressure-impedance data pairs. In some cases, the LCP may be configured to record a patent's metabolic demands, activity level, or other contextual data.

In some instances, the LCP may be in wireless communication with an external wearable device, such as an activity tracker (e.g. iWatch®, FitBit®, etc.), that may provide contextual information such as sleep status and/or activity level. It is contemplated that to the generated PP loops and/or VV loops may be processed or grouped according to context. For example, PP loops and/or VV loops may be processed according to time of day, posture, activity level, metabolic demands, heart rate range, sleep status, etc.

Those skilled in the art will recognize that the present disclosure may be manifested in a variety of forms other than the specific examples described and contemplated herein. For instance, as described herein, various examples include one or more modules described as performing various functions. However, other examples may include additional modules that split the described functions up over more modules than that described herein. Additionally, other examples may consolidate the described functions into fewer modules. Accordingly, departure in form and detail may be made without departing from the scope and spirit of the present disclosure as described in the appended claims.

What is claimed is:

1. A leadless cardiac pacemaker (LCP) system configured to sense cardiac activity and to pace a patient's heart, the system comprising:
a first leadless cardiac pacemaker (LCP), the first LCP comprising:
a housing;
a first electrode secured relative to the housing and exposed to the environment outside of the housing;
a second electrode secured relative to the housing and exposed to the environment outside of the housing, the second electrode is spaced from the first electrode;
a pressure sensor secured relative to the housing and is coupled to the environment outside of the housing; and
circuitry in the housing in communication with the first electrode, the second electrode, and the pressure sensor, the circuitry configured to determine at a first time during a cardiac cycle a first pressure via the pressure sensor;
a second LCP, the second LCP comprising:
a housing;
a first electrode secured relative to the housing and exposed to the environment outside of the housing;
a second electrode secured relative to the housing and exposed to the environment outside of the housing, the second electrode is spaced from the first electrode;
a pressure sensor secured relative to the housing and is coupled to the environment outside of the housing; and
circuitry in the housing in communication with the first electrode, the second electrode, and the pressure sensor the circuitry configured to determine at the same first time as the circuitry in the first LCP a first pressure via the pressure sensor to generate a first pressure-pressure data pair.

2. The LCP system of claim 1, wherein at least one of the circuitry of the first or second LCP is configured to wirelessly transmit the first pressure-pressure data pair to a remote device.

3. The LCP system of claim 1, wherein the circuitry of the first and second LCP are each configured to determine, at a second time during the cardiac cycle, a second pressure via the corresponding pressure sensor, resulting in a second pressure-pressure data pair.

4. The LCP system of claim 3, wherein the first time corresponds to an S₁ heart sound and the second time corresponds to an S₂ heart sound.

5. The LCP system of claim 3, wherein the circuitry of the first and second LCP are each further configured to determine, at a plurality of times between the first time and the second time, a plurality of corresponding pressures via the corresponding pressure sensor, resulting in a plurality of additional pressure-pressure data pairs.

6. The LCP system of claim 5, wherein at least one of the circuitry of the first or second LCP is further configured to wirelessly transmit the first pressure-pressure data pair, the second pressure-pressure data pair, and the plurality of additional pressure-pressure data pairs to a remote device.

7. The LCP system of claim 6, wherein the remote device is configured to generate and display a pressure-pressure loop that is based at least in part on the first pressure-pressure data pair, the second pressure-pressure data pair, and the plurality of additional pressure-pressure data pairs.

8. The LCP system of claim 7, wherein the remote device is configured to store and display a plurality of pressure-pressure loops generated over a period of time.

9. The LCP system of claim 1, wherein at least one of the circuitry of the first or second LCP is further configured to record contextual data indicating the patient's physiological state present during the cardiac cycle.

10. The LCP system of claim 9, wherein the indication of the patient's physiological state comprises one or more of a level of activity, sleep/wakefulness, a disease status, an effect of a therapy, a heart rate, a respiratory rate, a blood gas, a blood analyte, or a posture.

11. The LCP system of claim 1, wherein the circuitry of the first and second LCP are each further configured to average the first pressures over a plurality of cardiac cycles, resulting in an averaged first pressure-pressure data pair for the first time during an averaged cardiac cycle.

12. The LCP system of claim 5, wherein:
the circuitry of the first LCP is configured to determine an impedance between the first and second electrodes of the first LCP at each of a plurality of times during the cardiac cycle;
the circuitry of the second LCP is configured to determine an impedance between the first and second electrodes of the second LCP at each of the plurality of times during the cardiac cycle to generate a plurality of impedance-impedance data pairs; and
wherein at least one of the circuitry of the first or second LCP is configured to wirelessly transmit the plurality of impedance-impedance data pairs to a remote device.

13. A leadless medical device comprising:
a housing;
a first electrode secured relative to the housing and exposed to the environment outside of the housing;
a second electrode secured relative to the housing and exposed to the environment outside of the housing, the second electrode is spaced from the first electrode;
a pressure sensor secured relative to the housing and is coupled to the environment outside of the housing; and
circuitry in the housing in operatively coupled to the first electrode, the second electrode, and the pressure sensor, the circuitry configured to determine a pressure via the pressure sensor at a plurality of times during a cardiac cycle of a patient to generate a plurality of time/pressure pairs that correspond to the cardiac cycle, the circuitry further configured to wirelessly communicate the plurality of time/pressure pairs to a remote device via the first electrode and the second electrode.

14. The leadless medical device of claim 13, wherein the circuitry receives a synchronization marker from the remote device, and the plurality of times are expressed relative to the synchronization maker.

15. The leadless medical device of claim 13, wherein the circuitry further wirelessly communicates a synchronization marker to the remote device, and the plurality of times are expressed relative to the synchronization maker.

16. A method, comprising:
receiving a measure of right ventricle pressure from a first leadless medical device implanted in the right ventricle (RV) of a patient's heart and a measure of left ventricle pressure from a second leadless medical device implanted in the left ventricle (LV) of the patient's heart for each of a plurality of times during a cardiac cycle; and
generating a pressure-pressure loop that is based at least in part on the measure of right ventricle pressure and the measure of left ventricle pressure for each of the plurality of times during the cardiac cycle; and
displaying the pressure-pressure loop on a display and/or altering an operation of the first leadless medical device and/or the second leadless medical device based at least in part on the pressure-pressure loop.

17. The method of claim 16, wherein the first leadless medical device and the second leadless medical device are leadless cardiac pacemakers, and the method further comprises changing one or more pacing parameters of the first leadless medical device and/or one or more pacing parameters of the second leadless medical device to improve synchronization between the RV and the LV of the patient's heart.

18. The method of claim 16, wherein the first leadless medical device and the second leadless medical device are leadless cardiac pacemakers, and the method further comprises changing an in implant site of the first leadless medical device and/or the implant site of the second leadless medical device to improve synchronization between the RV and the LV of the patient's heart.

19. The method of claim 16, wherein the cardiac cycle is an averaged cardiac cycle, and the measure of right ventricle pressure and the measure of left ventricle pressure are averaged pressures for each of the plurality of times during the averaged cardiac cycle.

20. The method of claim 16, further comprising receiving contextual data indicating a patient's physiological state present during the cardiac cycle, and wherein the patient's physiological state comprises one or more of a level of activity, sleep/wakefulness, a disease status, an effect of a therapy, a heart rate, a respiratory rate, a blood gas, a blood analyte, or a posture.
